(19) United States
(12) Patent Application Publication
(10) Pub. No.: US 2009/0105281 A1
(43) Pub. Date: Apr. 23, 2009

(54) Methods of treating inflammation

(76) Inventors: Paul Epstein, Weatogue, CT (US); Stefan Brocke, West Hartford, CT (US); Hongli Dong, Glastonbury, CT (US); Amanda Dall, Farmington, CT (US)

Correspondence Address: MINTZ, LEVIN, COHN, FERRIS, GLOVSKY AND POPEO, P.C, ONE FINANCIAL CENTER, BOSTON, MA, 02111, US

(21) Appl. No.: 11/796,259

(22) Filed: Apr. 27, 2007

Related U.S. Application Data
(60) Provisional application No. 60/795,652, filed on Apr. 27, 2006.

(51) Int. Cl. A61K 31/519; A61P 29/00; A61P 3/10; A61P 25/00; A61P 35/00; C12Q 1/02; C12Q 1/34; C12Q 1/68

(52) U.S. Cl. 514/262.1; 435/29; 435/18; 435/6

(57) ABSTRACT

The invention features compositions and methods for treating inflammation and other immune-related disorders.

### RELATED APPLICATIONS

This application claims the benefit of U.S. Provisional Application No. 60/795,652, filed Apr. 27, 2006, the contents of which are hereby incorporated by reference in their entirety.

### FIELD OF THE INVENTION

This invention relates to compositions and methods for treating inflammation and other immune-related disorders.

### BACKGROUND OF THE INVENTION

Inflammation is the complex biological response of vascular tissues to harmful stimuli, such as pathogens, damaged cells, or irritants.

Inflammation can be classified as either acute or chronic. Acute inflammation is the initial response of the body to harmful stimuli and is achieved by the increased movement of plasma and leukocytes from the blood into the injured tissues. A cascade of biochemical events propagates and matures the inflammatory response, involving the local vascular system, the immune system, and various cells within the injured tissue. Prolonged inflammation, known as chronic inflammation, leads to a progressive shift in the type of cells which are present at the site of inflammation and is characterized by simultaneous destruction and healing of the tissue from the inflammatory process.

Commonly, inflammation occurs as a defensive response to invasion of the host by foreign, particularly microbial, material. Responses to mechanical trauma, toxins, and neoplasia also may results in inflammatory reactions. The accumulation and subsequent activation of leukocytes are central events in the pathogenesis of most forms of inflammation. Deficiencies of inflammation compromise the host. Excessive inflammation caused by abnormal recognition of host tissue as foreign or prolongation of the inflammatory process may lead to inflammatory diseases as diverse as diabetes, arteriosclerosis, cataracts, reperfusion injury, and cancer, to post-infectious syndromes such as in infectious meningitis, rheumatic fever, and to rheumatic diseases such as systemic lupus erythematosus and rheumatoid arthritis. The centrality of the inflammatory response in these varied disease processes makes its regulation a major element in the prevention control or cure of human disease.

Accordingly, there exists a need for methods of treating inflammation and other immune-related disease.

### SUMMARY OF THE INVENTION

The present invention comprises methods of treating diseases of the immune system, particularly chronic diseases of the immune system, and more particularly, autoimmune diseases such as multiple sclerosis (MS). The invention features a method of inhibiting migration of an activated lymphocyte to a site of inflammation by contacting the lymphocyte with a composition that inhibits cyclic nucleotide phosphodiesterase (PDE) 8. The composition inhibits activated lymphocytes as well as unstimulated or unactivated lymphocytes. The composition preferentially inhibits PDE8 compared to other cellular PDEs such as PDE 1, 2, 3, 4, 5, 6, 7, 9, 10, or 11. For example, the inhibitor reduces PDE activity or expression by at least 10%, 25%, 50%, 100%, 3-fold, 5-fold, 10-fold or more compared to another PDE such as PDE4. The activated lymphocyte or population of lymphocytes contains activated cells of the T helper type 1 phenotype and/or T helper 17 phenotype. An example of a preferential PDE8 inhibitor is dipyridamole or an derivative thereof. As defined herein, the term “derivative”, refers to compounds that have a common core structure, and are substituted with any of a variety of substituents. Other suitable PDE8A inhibitors for use in the anti-inflammatory composition and methods provided herein include PDE8A inhibitors such as E-4021 and papaverine (Soderling, et al., Proc Natl Acad Sci USA, vol. 95: 8991-8996 (1998); and Gamanuma et al., Cellular Signalling, vol. 15:565-574 (2003)). Optionally, the composition also contains an inhibitor of PDE4.

Also within the invention is an anti-inflammatory composition containing a combination of a PDE8 inhibitor and a PDE4 inhibitor or a PDE8 inhibitor and a PDE7 inhibitor. The combination of a PDE8 inhibitor and a second PDE inhibitor such as a PDE4 inhibitor or PDE7 inhibitor is a synergistic combination. The combination optionally includes inhibitors of PDE8, PDE4, and PDE7. Exemplary PDE4 inhibitors include Cilomilast (Ariflo, SB 207499, SmithKline Beecham), Roflumilast, PLX 369, PLX 743 (Plexxikon, Inc.), N-(3,5-Dichloro-pyrid-4-yl)-[1-(4-fluorobenzyl)-5-hydroxy-indole-3-yl]-glyoxylic acid amide (AWD 12-281), mesembrine (an alkaloid present in the herb, Sceletium tortuosum), and rolipram. However, their clinical use is limited due to adverse side effects. Exemplary PDE7 inhibitors include BRL 50481 [3-(N,N-dimethylsulfonamido)-4-methyl-nitrobenzene] (Smith et al., Mol. Pharmacol., vol. 66(6): 1679-1689 (2004)); IC242 (Lee et al., Cell Signal, vol. 14:277-284 (2002)); T-2585 (Nakata, et al., Clin Exp Immunol., vol. 128(3): 460-6 (2002)), YM-393059 (Yamamoto et al., Eur J. Pharmacol., vol. 541(1-2):106-14 (2006); and Yamamoto et al., Eur J. Pharmacol. Vol. 559(2-3): 219-26 (2007)), a series of compounds such as BMS-586353 described in Lorthiois et al., Bioorg. Med. Chem. Lett., vol. 14: 4623-26 (2004); Pitts et al., Bioorg. Med. Chem. Lett., vol. 14: 2955-58 (2004) and Vergne et al., Bioorg. Med. Chem. Lett., vol. 14:4607-13 (2004)); and 8-bromo-9-substituted derivatives of guanine (Barnes et al., Bioorg. Med. Chem. Lett., vol. 14:1081-83 (2001)). The co-administration compositions include, for example, compounds that are dual PDE4 and PDE7 inhibitors, such as, for example, T-2585 and YM-393059

Co-administration of a PDE4 inhibitor (and/or a PDE7 inhibitor) and a PDE8 inhibitor provides a synergistic anti-inflammatory effect compared to administration of PDE4 (or PDE7) alone or PDE8 alone. The combination is more efficacious with the added advantage of reduced adverse side effects associated with anti-inflammatory amounts of PDE4 alone. The combination optionally includes other anti-inflammatory agents such as inhibitors of other PDEs or any of a variety of known anti-inflammatory agents such that the efficacy of the known anti-inflammatory agent is enhanced by the combination and/or the therapeutically effective amount of the known anti-inflammatory agent is reduced when used in combination with a PDE inhibitor of the invention. Suitable anti-inflammatory agents for use in such combination therapy include, for example, glucocorticoids, anti-adhesion antibodies such as anti-alpha 4 integrin antibodies, e.g., Tysabri, interferons, and antagonists of TNF-alpha such anti-TNF-alpha antibodies, e.g., Humira, Remicade and Enbrel. The synergistic combination is more effective in inhibiting lymphocyte migration or adhesion and reducing inflammation than either ingredient alone.

A method of reducing or preventing a symptom associated with a disorder of the immune system is carried out by administering to a subject in need thereof a composition containing an inhibitor of PDE8 in an amount effective to modulate, e.g., reduce, lymphocyte chemotaxis. The methods described herein are useful for administration to humans as well as other animals (e.g., dogs, cats, horses, cattle, sheep, pigs) that are identified as suffering from or at risk of developing an autoimmune or inflammatory condition/disorder. For example, the disease state is selected from the group comprising multiple sclerosis, rheumatoid arthritis, asthma, and inflammatory bowel disease. Optionally, the composition further contains an inhibitor of PDE4. The compounds are formulated together in one composition or are formulated individually. The PDE8 and PDE4 inhibitory compounds are co-administered or administered in sequence, i.e., administration of one inhibitor occurs before or after administration of the other inhibitor. The compositions are administered systemically, e.g., orally, intravenously, intramuscularly, or locally, e.g., topically, to a site of inflamed tissue.

In one example, the disease is multiple sclerosis, an autoimmune disease characterized by inflammation and an immune response directed against myelin of nerve fibers, brain or spinal cord. The inhibitor of PDE8 is dipyridamole or a clinically effective derivative thereof. Inhibitory compounds are administered during an episode to reduce pain and neurological symptoms or compositions are administered during a period of remission to prevent the occurrence of an episode and/or to reduce the severity of a subsequent episode of multiple sclerosis.

The compositions and methods are useful to modulate, e.g., reduce an inflammatory response, in a subject. The PDE8 or PDE8/4 inhibitory compositions are administered to a subject in need thereof in an amount effective to modulate, e.g., reduce, lymphocyte chemotaxis in the subject thereby treating or ameliorating the symptoms of a disease associated with lymphocyte chemotaxis, lymphocyte adhesion to endothelial cells, and/or lymphocyte transendothelial migration. Thus, the compounds are administered in an amount effective to modulate, e.g., reduce, lymphocyte chemotaxis, lymphocyte adhesion to endothelial cells, and/or lymphocyte transendothelial migration in subject. Exemplary disorders to be treated also include an autoimmune disease or allergic disease selected from the group comprising multiple sclerosis, type 1 diabetes, rheumatoid arthritis, asthma, chronic obstructive pulmonary diseases, inflammatory bowel disease, Alzheimer's disease and other neurodegenerative diseases with inflammatory components, atherosclerosis, vasculitis, and cancer, such as metastatic cancers. Other disorders or conditions to be treated include inflammatory diseases and conditions such as joint inflammation, rheumatoid arthritis, rheumatoid spondylitis, osteoarthritis, chronic glomerulonephritis, dermatitis, and inflammatory bowel disease, such as, for example, ulcerative colitis and/or Crohn's disease; respiratory diseases and conditions such as asthma, acute respiratory distress syndrome, chronic pulmonary inflammatory disease, bronchitis, chronic obstructive airway disease, and silicosis; infectious diseases and conditions such as sepsis, septic shock, endotoxic shock, gram negative, sepsis, toxic shock syndrome, fever and myalgias due to bacterial, viral or fungal infection, and influenza; Alzheimer's disease and other neurodegenerative diseases with an inflammatory component immune diseases and conditions such as autoimmune diabetes, systemic lupus erythematosis, graft vs. host reaction, allograft rejections, multiple sclerosis, psoriasis, and allergic rhinitis. Administration of PDE8 inhibitors or a synergistic combination of PDE8/4 inhibitors also confer clinical benefit to those suffering from other diseases and conditions such as bone resorption diseases; reperfusion injury; cachexia secondary to infection or malignancy; cachexia secondary to human acquired immune deficiency syndrome (AIDS), human immunodeficiency virus (HIV) infection, or AIDS related complex (ARC); keloid formation; scar tissue formation; type 1 diabetes mellitus; or leukemia.

The compositions and methods are useful in treating, reducing the spread of, delay the progression of or otherwise preventing, migration of metastatic cancer cells. Thus, the selective PDE inhibitors identified herein are useful in modulating, e.g., reducing the migration of metastatic cancer cells. Compositions useful in methods of reducing the migration of metastatic cancer cells are delivered systematically by administering these compositions to a subject in need thereof. These compositions, for example, are formulated for oral administration. Compositions useful in methods of treating or alleviating a symptom of a metastatic cancer are administered, for example, as an adjunct therapy in addition to other known chemotherapy agents.

For example, the invention provides methods of inhibiting migration of a metastatic cancer cell by contacting the metastatic cancer cell with a composition that preferentially inhibits cyclic nucleotide phosphodiesterase (PDE) 8. For example, the metastatic cancer cell is derived from epithelial tissue. The composition includes, for example, a second agent such as an inhibitor of PDE4, an inhibitor of PDE7 or both an inhibitor of PDE4 and an inhibitor of PDE7. The inhibitor of PDE8 is, for example, dipyridamole or a clinically effective derivative thereof.

The invention also provides methods of treating or delaying the progression of a metastatic cancer by administering to a subject in need thereof a composition that includes an inhibitor of PDE8 in an amount effective to reduce migration of metastatic cancer cells. For example, the metastatic cancer cell is derived from epithelial tissue. The composition includes, for example, a second agent such as an inhibitor of PDE4, an inhibitor of PDE7 or both an inhibitor of PDE4 and an inhibitor of PDE7. The inhibitor of PDE8 is, for example, dipyridamole or a clinically effective derivative thereof.

The methods include a step of diagnosing and/or identifying a subject comprising a primary tumor selected from the group consisting of colorectal, stomach, pancreatic, biliary tree, small intestine, kidney, breast, prostate, ovarian, malignant melanoma, lung cancer, and lymphoma. These cells of these primary tumor types are characterized by metastatic migration mediated by chemokines such as CXCL12.

The subset of cancers known as “metastatic cancers” refer to those types of cancers in which the primary tumor is prone to spread from its original site to another part of the subject, a process known as metastasizing. In metastatic cancers, the primary tumor is often an epithelial-derived tumor. Epithelial derived tumor types that are prone to metastasizing include primary tumors originating in the colon and rectum, stomach, pancreas, biliary tree, small intestine, kidney, breast, prostate, ovaries, malignant melanoma, lung cancer, and lymphoma. The compositions and methods are useful in treating and/or preventing these metastatic cancers by inhibiting or otherwise reducing the migration of the metastatic cancer cells from the original site of the primary tumor to a second site within a subject.

Methods of identifying and/or diagnosing an individual who is suffering from or is at risk of developing a metastatic cancer are known in the art. For example, detection of a serum marker associated with metastatic cancer in an individual indicates that the individual is, has, or is at risk of developing metastases. CT scan or ultrasound is also used to confirm the presence of a tumor in any of the tissues and organs listed above. Diagnosis of any one of the above-listed primary tumors indicates that an individual is at risk of developing a metastasis. Multiple metastatic lesions are often the case, but single metastases may be seen. Optionally, biopsy is carried out to confirm metastatic cancer.

The compositions and methods are also useful in treating, alleviating a symptom of, delaying the progression of or otherwise preventing cancers that are induced by chronic inflammation. For example, the compounds and methods are useful in the treatment of a carcinoma. These compositions and methods are useful in the treatment of a cancer derived from epithelial tissues. For example, the compositions and methods are useful in the treatment of colon cancer and/or liver cancer. Compositions useful in methods of treating or alleviating a symptom of cancers induced by chronic inflammation are administered, for example, as an adjunct therapy in addition to other known chemotherapy agents.

The methods are also useful in reducing the symptoms of respiratory disorders such as allergen-induced or inflammation-induced bronchial disorders such as bronchitis, obstructive bronchitis, spastic bronchitis, allergic bronchitis, allergic asthma, bronchial asthma, and chronic obstructive pulmonary disease (COPD) as well as inflammatory conditions of the gastrointestinal tract or bowel.

Those of ordinary skill in the art will appreciate that the dosages and formulations for the administration of a selective PDE inhibitor, alone or in combination with other selective PDE inhibitors, is determined based on the variables such as the potency of the inhibitor, the formulation, and the route of administration. The dosages used in the methods and co-therapy methods do not exceed the dosage at which the PDE inhibitor ceases to be a selective for a given PDE gene family. For example, Rolipram, the prototypical PDE4 inhibitor, will for example lose its specificity above 10 μM.

In the methods provided herein, the selective PDE8 inhibitor is administered at a dosage in the range of 0.01 to 100 mg/kg/day. The PDE8 inhibitor is administered at a dosage in the range of 0.1 to 10 mg/kg/day. For example, the PDE8 inhibitor is administered at dosage selected from 5 mg/kg/day, 3 mg/kg/day, 2.5 mg/kg/day, 2 mg/kg/day, 1.5 mg/kg/day, 1 mg/kg/day, 0.75 mg/kg/day and 0.5 mg/kg/day.

In the methods provided herein, the dosage of the PDE4 inhibitor depends on the inhibitor used. For example, roflumilast is administered at a dosage in the range of 100 μg/day to 1 mg/day, and preferably in the range of 250 μg/day to 500 μg/day. For example, roflumilast is administered once daily at a dosage of 500 μg/day. PDE4 inhibitors such as cilomilast are administered at a dosage between 1 mg/kg/day and 100 mg/kg/day and preferably at a dosage between 10 mg/kg/day and 30 mg/kg/day. For example, PDE4 inhibitors such as cilomilast are administered twice daily at a dosage in the range of 5 mg/kg to 15 mg/kg. (Lipworth, Lancet, vol. 365:167-75 (2005)).

A further object of the present invention is to treat diseases of the immune system at the level of regulating and/or controlling the migration and subsequent action, such as adhesion, of activated lymphocytes. The invention also comprises methods of regulating and/or controlling, primarily methods of inhibiting, the recruitment of activated lymphocytes to sites of inflammation to thereby decrease the severity and/or duration of such inflammation. Inflammatory disorders include, for example, chronic and acute inflammatory disorders.

The invention comprises the use of inhibitors of one or more cyclic nucleotide phosphodiesterases (PDE) to reduce or block the migration of activated lymphocytes, and thereby reduce the subsequent adhesion and/or infiltration of such lymphocytes. The PDEs comprise a family of related enzymes encoded by at least 21 different genes, grouped into 11 different gene families (PDEs # 1 to 11).

Of the PDEs, PDE4, PDE7 and PDE8 are known to be present in mouse and human lymphocytes. Certain of these PDEs, such as PDE4, have been shown to be induced during lymphocyte activation. It has further been shown that inhibition of PDE4 alone is not sufficient to effectively block the migration of such activated lymphocytes.

The present invention demonstrates that inhibition of PDE8 is also needed in order to block migration and subsequent actions of activated lymphocytes. The invention further demonstrates in a clinical experiment in mammals that inhibition of PDE8 results in a profound decrease in the observed symptoms, such as paralytic signs, in a model mammalian system for experimental autoimmune encephalitis (EAE). It is a further object of the present invention to use methods for inhibiting PDE8 as methods of treating inflammatory autoimmune or allergic diseases such as multiple sclerosis, rheumatoid arthritis, asthma and inflammatory bowel disease.

The invention provides methods of modulating, e.g., treating, reducing, alleviating or otherwise preventing, inflammation or other immune-related diseases by administering selective PDE inhibitors, and preferably, at least a selective PDE8 inhibitor. The selective PDE8 inhibitor is, for example, a commercially available selective inhibitor, such as dipyridamole. Other suitable selective PDE8 inhibitors include compounds identified by screening chemical libraries for novel compounds that inhibit PDE8 with the same or better ability as dipyridamole. Other suitable selective PDE8 inhibitors are created, for example, through rational design. In one embodiment, the selective PDE8 inhibitor is rationally designed to have a structure that is based on the structure of dipyridamole or a derivative thereof.

Selective inhibitors of PDE8 are administered alone or in combination with other suitable therapeutic agents. For example, the selective PDE8 inhibitor is administered in combination with one or more additional PDE inhibitors, such as, a PDE4 inhibitor.

The invention provides methods of modulating, e.g., treating, reducing, alleviating or otherwise preventing, inflammation or other immune-related diseases by administering a selective inhibitor that targets a novel PDE isoform that is identified using the methods provided herein. Suitable selective PDE inhibitors include, for example, a commercially available selective inhibitor, compounds identified by screening known chemical libraries for novel compounds that inhibit the novel PDE isoform, and inhibitors created through rational design.

Targeting PDE8 and other novel PDE isoforms maximizes the therapeutic potential in the treatment of inflammation, while simultaneously increasing the therapeutic index of PDE inhibition. Targeting of these PDE isoforms therefore overcomes the limitations observed in human anti-inflammatory therapies with selective PDE4 inhibitors.

The selective PDE inhibitors used in the methods of the invention, such as, the selective PDE8 inhibitors, are administered in an amount that is effective to treat, reduce, alleviate or otherwise prevent multiple sclerosis and other autoimmune diseases associated with chemokine-induced migration of leukocytes.

In addition to targeting pro-inflammatory T effector cells, the selective PDE inhibitors are also useful in treating, alleviating, delaying the progression of, or otherwise preventing inflammatory diseases through immune deviation or induction of immunosuppressive regulatory T cells (Tregs) in vivo. Studies have shown that signals required to induce and maintain Tregs include Foxp3-dependent repression of PDE3B (Gavin, et al., Nature, ePublication, (Jan. 14, 2007)). A PDE inhibitor such as a PDE8 inhibitor contributes to regulatory T cell development and function, and is, therefore, effective in the treatment of inflammatory diseases (Qiao, et al., Immunology, vol. 120(4):447-55 (2007); Shevach et al., Immunol Rev 212:60-73 (2006).

The invention also provides methods of identifying putative selective PDE inhibitors, such as, for example, PDE8 inhibitors by determining the ability of a test compound to inhibit the migration of stimulated and unstimulated splenocytes, for example, using the chemotaxis assays disclosed herein. Screening methods to identify anti-inflammatory compositions to inhibit migration and/or adhesion of activated lymphocytes are carried out as follows. A method of identifying a selective PDE8 inhibitor composition is carried out by contacting an activated lymphocyte with a candidate PDE inhibitory compound and detecting migration of the activated lymphocyte. The assay is carried out using a population of stimulated lymphocytes or a mixed population of stimulated and unstimulated cells. Optionally, the cells are genetically modified, e.g., the cells lack expression of one or more PDEs (e.g., cells from a PDE−/− knockout mouse). A reduction in migration of an activated lymphocyte compared to migration of an unstimulated lymphocyte indicates that the candidate compound selectively inhibits a PDE8-mediated anti-inflammation.

Another method of identifying a selective PDE8 inhibitor composition includes the following steps: contacting an activated lymphocyte with a candidate PDE inhibitory compound and detecting migration of the activated lymphocyte, wherein a reduction in migration of the activated lymphocyte in the presence of the compound compared to in the absence of the compound indicates that the candidate compound inhibits a PDE8-mediated inflammation.

Another method of identifying a selective PDE8 inhibitor composition includes the following steps: contacting an activated lymphocyte with a candidate PDE inhibitory compound and detecting expression or activity of PDE8 in the activated lymphocyte. A reduction in PDE 8 expression or activity compared to a PDE selected from the group consisting of PDE1, 2, 3, 4, 5, 6, 7, 9, 10, or 11 indicates that the candidate compound selectively inhibits a PDE8-mediated anti-inflammation.

The invention also provides methods of identifying putative selective PDE inhibitors, such as PDE8 inhibitors, by determining the ability of a test compound to inhibit one or more effector functions of an inflammatory cell population. For example, putative PDE inhibitors are identified by their ability to inhibit proliferation, cytotoxicity and/or cytokine production such as the production of interleukin-2, interferon gamma, interleukin-17 and/or TNF-alpha. The level of effector function in the presence or absence of a test compound is identified, for example, using standard assays including ELISA assays.

Other features, objects, and advantages of the invention will be apparent from the description and drawings.

### BRIEF DESCRIPTION OF THE DRAWINGS

FIG. 1 is a graph depicting the effect of CXCL12 on migration of splenocytes.

FIG. 2 is a graph depicting the effect of dibutyryl cAMP on splenocyte chemotaxis in response to CXCL12.

FIG. 3 is a graph depicting the effect of forskolin and IBMX on splenocyte chemotaxis in response to CXCL12.

FIG. 4 is a graph depicting the effect of PDE gene family-specific inhibitors on chemotaxis of stimulated splenocytes in response to CXCL12.

FIG. 5 is a graph depicting the effect of dipyridamole on splenocyte chemotaxis in response to CXCL12.

FIGS. 6A and 6B are a series of graphs depicting the effect of Rp-cAMPS and adenosine deaminase on dipyridamole inhibition of splenocyte chemotaxis in response to CXCL12.

FIG. 7 is a graph depicting the expression of mRNA for PDE8A1, PDE4B2 and PDE7A1 following Con A stimulation.

FIG. 8 is a graph depicting the effect of dipyridamole (DP) treatment on experimental autoimmune encephalomyelitis (EAE).

FIG. 9 is an illustration depicting the role of PDEs in regulation of signal transduction.

FIG. 10 is a graph depicting the effect of PDEs isoforms on the regulation of cAMP/PKA signaling in T cells.

FIGS. 11A and 11B are a series of graphs depicting the effect of PDE8 on splenocyte adhesion to a bEND.3 monolayer. In FIG. 11A, Con A activated splenocytes were adhered to bEND.3 cells in the presence of dipyridamole (100 μM) or piclamast (1 μM).

FIG. 12 is a graph depicting the suppression of inflammation by dipyridamole.

FIG. 13 is an illustration depicting the experimental scheme for the identification of novel PDE targets in stimulated T cells treated with selective PDE inhibitors.

FIG. 14 is an illustration depicting the experimental scheme for determining regulatory functions of novel PDE isoforms in anti-CD3 and anti-CD28 stimulated cells and T cells treated with selective PDE inhibitors.

FIG. 15 is an illustration depicting recruitment steps I-IV (I, rolling; II, adhesion; III, locomotion; IV, transendothelial migration (TEM)) as measured in the flow chamber assay by real-time videomicroscopy.

FIG. 16 is an illustration depicting the experimental scheme for analyzing the individual and overlapping functions of PDE1, PDE3, PDE4, PDE7 and PDE8 gene families during inflammation.

FIG. 17 is an illustration depicting the experimental scheme for determining the effect of PDE7 inhibitors and/or PDE8 inhibitors on the dosage requirements of PDE4 inhibitors in an EAE model in vivo.

FIG. 18 is a schematic illustration of the PDE8A1 protein.

### DETAILED DESCRIPTION

The methods provided herein employ PDE inhibitor therapy to modulate, e.g., reduce, inhibit, treat or prevent inflammatory illnesses and other immune-related diseases. The immune system depends on chemokines to recruit lymphocytes to tissues in inflammatory diseases. Thus, the invention provides methods of modulating inflammatory diseases by inhibiting migration of activated lymphocytes using selective PDE inhibitors. The Examples provided herein identified phosphodiesterase 8 (PDE8) as a target for inhibition of chemotaxis of activated lymphocytes. In particular, chemotactic responses of unstimulated and concanavalin A-stimulated mouse splenocytes and their modulation by agents that stimulate the cAMP signaling pathway were compared. Dibutyryl cAMP inhibited migration of both cell types. In contrast, forskolin and 3-isobutyl-1-methylxanthine each inhibited migration of unstimulated splenocytes, with little effect on migration of stimulated splenocytes. Dipyridamole alone, a PDE inhibitor capable of inhibiting PDE8, strongly inhibited migration of stimulated and unstimulated splenocytes, and this inhibition was enhanced by forskolin and reversed by a PKA antagonist. Following concanavalin A stimulation, mRNA for PDE8A1 was induced.

Thus, the methods provided herein modulate, e.g., inhibit migration of activated lymphocytes using selective inhibitors of PDE8. The invention also provides methods of identifying other PDE isoforms as targets for PDE inhibitor therapy. Thus, the methods provided herein also include methods of modulating, e.g., reducing, inhibiting, treating or preventing inflammatory illnesses and other immune-related diseases by administering inhibitors of these identified PDE isoforms to reduce or block the migration of activated lymphocytes, and thereby reducing the subsequent adhesion and/or infiltration of such lymphocytes.

Phosphodiesterases (PDEs) are a family of related enzymes codes by at least 21 different genes, grouped into 11 different gene families (PDEs # 1 to 11). The data provided herein demonstrated that cyclic nucleotide phosphodiesterases (PDEs) expressed in activated T cells contribute to the control of effector T cell functions and thereby serve, in concert with PDE4, as targets for the treatment of inflammation. T cell functions and inflammation are tested in mice in which the genes for each of the PDEs present in activated T cells are deleted or inhibited pharmacologically through the use of selective PDE inhibitors both in vitro and in vivo.

Extravasation of T cells in post-capillary venules plays an important role in the pathogenesis of various inflammatory conditions. Thus, uncovering a means of inhibiting T cell recruitment and effector functions as described herein provided the basis for an effective treatment of inflammatory illnesses. Observations that PDE4 is the most abundantly expressed form of PDE in T cells, and that inhibition of PDE4 blocks T cell activation and function through elevating cAMP, generated considerable interest in developing pharmacological inhibitors of PDE4 as potential therapies for treatment of chronic inflammatory diseases, including chronic obstructive pulmonary disease (COPD). It is now accepted that individual PDE isoforms serve to modulate distinct regulatory pathways in cells, including T cells (Conti and Beavo., Annu Rev Biochem., ePublication (Mar. 21, 2007)). These properties therefore offer the opportunity for selectively targeting specific PDEs for treatment of specific disease states. However, despite high expectations for PDE4 inhibitors to treat inflammatory illnesses, when used in clinical trials, PDE4 inhibitors were less efficacious than preclinical data suggested; consequently, none has been approved for clinical use. Further, PDE4 inhibitors exhibit a low therapeutic index due to dose-limiting side effects which hampered their clinical development (Burnouf and Pruniaux, Curr Pharm Des, vol. 8:1255-1296 (2002); Giembycz, Proc Am Thorac Soc, vol. 2:326-333; discussion 340-321 (2005); Giembycz, Curr Opin Pharmacol, vol. 5:238-244 (2005); Giembycz and Smith, Curr Pharm Des, vol. 12:3207-3220 (2006); and Bender and Beavo. Pharmacol Rev, vol. 58:488-520 (2006)). Recent reports have shown roles for PDEs other than PDE4 in controlling cAMP signaling in T cells. It was reported that PDE7 and possibly PDE8 are required for T cell activation and induced by CD3 and CD28 stimulation (Li et al., Science, vol. 283:848-851 (1999); and Glavas et al., Proc Natl Acad Sci USA, vol. 98:6319-6324 (2001)). Therefore, PDE4 inhibitors may have shown limited efficacy in clinical trials because important PDE isoforms induced in activated T cells were not targeted. The studies described herein identified PDE8 as a novel target for inhibition of T cell chemotaxis. The Examples provided herein also provide studies that are designed to translate this approach to effector T cells and inflammation as a prelude to developing comprehensive PDE inhibitor treatments for inflammatory disorders.

With this in mind, it was hypothesized that 1) selective isoforms of the PDE1, PDE3, PDE7, and PDE8 gene families are induced in activated T cells causing inflammation, 2) as a consequence of this, in addition to members of the PDE4 gene family that are constitutively expressed in T cells, these induced isoforms are critical regulators of T cell functions, and 3) in order to maximize the effectiveness of PDE inhibition for full control of accumulation of activated T cells in tissues, it is necessary to identify and inhibit PDE isoforms in addition to PDE4 that are important in controlling T cell function.

The studies are carried out to identify novel PDE targets in anti-CD3 and anti-CD28 stimulated T cells treated with selective PDE inhibitors and from wildtype and PDE mutant mice. Identification of these targets is accomplished by first analyzing in vitro the expression of isoforms of the PDE1, PDE3, PDE4, PDE7 and PDE8 gene families in T cells using quantitative real-time RT-PCR (qRT-PCR) and Western immunoblotting procedures. This is done in anti-CD3 and anti-CD28 stimulated T cells from wildtype (wt) as well as specific PDE gene knock out (PDE^{-/-}) mice and selective PDE inhibitor-treated T cells to detect compensatory changes in PDE isoform expression during T cell activation.

Studies are also carried to determine the regulatory functions of novel PDE isoforms in anti-CD3 and anti-CD28 stimulated T cells from wildtype and PDE mutant mice and T cells treated with selective PDE inhibitors. Functions of defined PDEs in regulating T cell activity are determined by testing T cells in proliferation and cytokine production assays. The role of members of the PDE1, PDE3, PDE4, PDE7 and PDE8 gene families in regulating cAMP-PKA-dependent vascular T cell recruitment is then determined by real time videomicroscopy measuring rolling and arrest, activation and adhesion strengthening, and transendothelial migration (TEM) under physiologic shear stress in vitro.

Unique and overlapping functions of PDE1, PDE3, PDE4, PDE7 and PDE8 gene families during inflammation in vivo are determined. To accomplish this, the susceptibility to experimental inflammation of wt mice is compared to specific PDE^{-/-} mice. Additionally, the therapeutic effect of pharmacologic inhibition of specific PDE gene families is tested using selective PDE inhibitors alone or in combination during experimental inflammation in vivo.

cAMP and T cell function

Studies in vitro and in vivo have shown that T cell proliferation as well as effector and regulatory functions can be modulated by cAMP (Conti and Beavo, Annu Rev Biochem., ePublication (Mar. 21, 2007); Giembycz, Curr Opin Pharmacol, vol. 5:238-244 (2005); Bender and Beavo. Pharmacol Rev, vol. 58:488-520 (2006); Asirvatham, et al., J Immunol, vol. 173:4806-4814 (2004); Gavin, et al., Nature, ePublication, (Jan. 14, 2007); vol. 445(7129):771-5 (2007); and Conti, et al. J Biol Chem, vol. 278:5493-5496 (2003). To be fully activated, CD4+ T cells need engagement of the T cell receptor (TCR)-CD3 complex and costimulation through molecules such as CD28. cAMP inhibits T cell function (Laudanna, et al., J Biol Chem, vol. 272:24141-24144 (1997)) and proliferation by exerting negative control on the TCR and costimulatory receptor pathways (Li et al., Science, vol. 283:848-851 (1999); and Glavas et al., Proc Natl Acad Sci USA, vol. 98:6319-6324 (2001)). Complete T cell activation is believed to result from a reduction in cAMP, releasing the negative hold (Conti and Beavo, Annu Rev Biochem., ePublication (Mar. 21, 2007) and Bender and Beavo. Pharmacol Rev, vol. 58:488-520 (2006)).

PDEs as Regulators of cAMP Signaling and Therapeutic Targeting

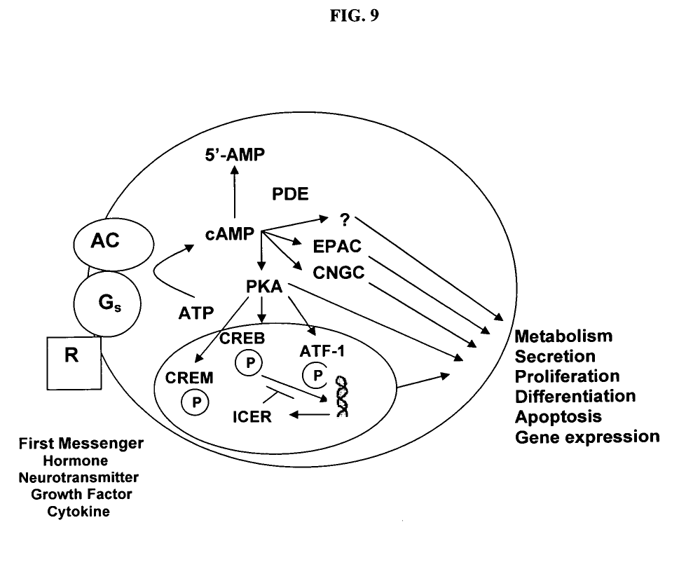

The second messenger cAMP plays important roles in mediating the biological effects of a wide variety of first messengers (Bender and Beavo. Pharmacol Rev, vol. 58:488-520 (2006); and Lerner and Epstein. Biochem J, vol. 393:21-41 (2006)). Increases in intracellular cAMP lead to activation of cAMP-dependent protein kinases, guanine nucleotide exchange factors, and cyclic nucleotide-gated channels, which in turn can regulate the activity of other signaling and metabolic pathways (FIG. 9). In the model of the second-messenger concept originally put forth by Sutherland and colleagues (shown in FIG. 9), first messengers, such as hormones, neurotransmitters, cytokines and growth factors, upon interacting with receptors on the cell surface, generate the production of a ‘second messenger’ such as cAMP, which then redirects the machinery of the cell, affecting many physiological processes. Currently, three different types of effector proteins to which cAMP can bind and carry out its actions are known: PKA (cAMP-dependent protein kinase), EPAC (exchange protein activated by cAMP) and CNGCs (cyclic nucleotide-gated channels). Activation of PKA by cAMP leads to changes both in cytosolic proteins and in gene transcription through phosphorylation of cAMP-responsive nuclear factors such as CREB, CREM (CRE modulator) and ATF-1 (activating transcription factor 1). PDEs, by regulating cAMP levels, play a central role in modulating all of these cAMP signalling pathways and consequent physiological responses. AC, adenylate cyclase; R, receptor (seven-membrane-spanning G-protein-coupled metabotropic receptor); Gs, G protein coupled to stimulation of adenylyl cyclase.

cAMP signaling pathways are controlled through regulation of the synthesis of cAMP by adenylyl cyclases and degradation by PDEs. The cyclic nucleotide-PDEs are now recognized to form a superfamily of 11 different, but homologous, gene-families that all contain highly homologous catalytic domains near their C termini and give rise to more than 50 protein products (Bender and Beavo. Pharmacol Rev, vol. 58:488-520 (2006); Lerner and Epstein. Biochem J, vol. 393:21-41 (2006); Soderling and Beavo. Curr Opin Cell Biol, vol. 12:174-179 (2000); and Francis, et al., Prog Nucleic Acid Res Mol Biol, vol. 65:1-52 (2001)). PDE-catalyzed cyclic nucleotide degradation provides an important mechanism for regulating signaling. Inhibition of PDEs reduces cAMP hydrolysis resulting in subsequent elevations in cAMP levels within the cell. Given the large number of different PDE isoforms now known to exist, and their selective expression and localization within cells and subcellular regions, it is now recognized that inhibition of different forms of PDE results in increases in cAMP in restricted microdomains within the cell (Mongillo, et al. Circ Res, vol. 95:67-75 (2004); Rich, et al., Proc Natl Acad Sci USA, vol. 98:13049-13054 (2001); and Rich, et al., J Gen Physiol, vol. 118:63-78 (2001)). Indeed, it is now acknowledged that the PDE component of cAMP pathways ensures the proper intensity and spatiotemporal distribution of the signal, as illustrated by many studies on different endocrine tissues (Conti, et al. J Biol Chem, vol. 278:5493-5496 (2003); and Conti, Mol Endocrinol, vol. 14:1317-1327 (2000)).

### Targeting PDEs

At the time of the discovery of PDE activity, it was also found that caffeine (1,3,7-trimethylxanthine), which is structurally similar to the substrate, cAMP, was an effective competitive inhibitor of PDE activity and a number of nonselective PDE inhibitors including the caffeine analog, theophylline (1,3-dimethylxanthine), have been in use as therapeutic agents for asthma and several other illnesses for decades (Bender and Beavo. Pharmacol Rev, vol. 58:488-520 (2006)). Thus, the principle that PDEs are valid targets and that inhibition of PDE activity is a valid therapeutic approach to treatment of inflammatory diseases is now well accepted. However, since the early PDE inhibitors were non-selective and targeted most known PDE isoforms, they had a very narrow therapeutic index. Nevertheless, one important reason that PDEs have been pursued as therapeutic targets is related to the basic pharmacological principle that regulation of degradation of any ligand or second messenger can often make a more rapid and larger percentage change in concentration than comparable regulation of the rates of synthesis. This is true for either pharmacokinetic changes in drug levels or changes in amounts of an endogenous cellular regulatory molecule or metabolite. Indeed, it has been found that almost all tissues contain at least an order of magnitude higher maximal PDE activity than cyclase activity for either cAMP or cGMP (Conti, et al. J Biol Chem, vol. 278:5493-5496 (2003)). It has been apparent for many years now that there is a rather extraordinarily large number of different forms of PDEs expressed in mammalian tissues, each of which can have a unique architecture at the active site. Moreover, there is increasing evidence that many of these PDEs are tightly connected to different physiological functions in the body and by inference also to different pathological conditions. Therefore, it has been widely believed that it should be possible to develop isoform selective inhibitors that can target specific functions and pathological conditions without a high likelihood of causing nonspecific side effects. The recent therapeutic and commercial success of agents such as sildenafil (Viagra), a selective PDE5 inhibitor, has validated the concept.

### PDEs and T Cells

It is well established that PDE3 (3B) and PDE4 (4A,B and D) constitute the major gene families and activity in T cells (Tenor, et al., Clin Exp Allergy, vol. 25:616-624 (1995); and Giembycz, et al., Br J Pharmacol, vol. 118:1945-1958 (1996)). However, recent evidence suggests that additional isoforms, including PDE1 (1B1, 1B2), PDE7 (7A1, 7A3), and PDE8 (8A1) are expressed and upregulated in T cells as a response to activation signals (Li et al., Science, vol. 283:848-851 (1999); Glavas et al., Proc Natl Acad Sci USA, vol. 98:6319-6324 (2001); Jiang, et al., Proc Natl Acad Sci USA, vol. 93:11236-11241 (1996); and Bender, et al., Proc Natl Acad Sci USA, vol. 102:497-502 (2005)). Thus, determining the spatiotemporal distribution and functional significance of the entire spectrum of PDEs expressed within effector/memory T cells remains a critical challenge to be addressed.

cAMP has long been known to inhibit lymphocyte activation, proliferation and function (Bourne, et al., Science, vol. 184:19-28 (1974); and Kammer, Immunol Today, vol. 9:222-229 (1988)). cAMP was also reported to inhibit motility of cytotoxic T lymphocytes (Valitutti, et al., Eur J Immunol, vol. 23: 790-795 (1993)). Inasmuch as recruitment of lymphocytes to sites of inflammation, i.e., their migration through postcapillary endothelial layers and subsequent parenchymal accumulation (Rot and von Andrian, Annu Rev Immunol, vol. 22: 891-928 (2004)) underlies the basis of a number of autoimmune diseases, such as multiple sclerosis (Engelhardt, and Ransohoff, Trends Immunol 26: 485-495 (2005); Steinman, Science 305: 212-216 (2004); and Steinman, Cell, vol. 85: 299-302 (1996)), stimulating the cAMP signaling pathway in lymphocytes as a means to inhibit their migration, has been suggested as the basis of very effective treatments for these diseases (Bielekova, et al., J Immunol, vol. 164:1117-1124 (2000)). cAMP levels in cells are controlled by their synthesis by adenylyl cyclases and degradation by cyclic nucleotide phosphodiesterases (PDEs). PDEs comprise a superfamily of related enzymes encoded by at least 21 different genes, grouped into 11 different gene families (PDEs 1-11), based on sequence similarity, mode of regulation and preference for cAMP or cGMP as substrate (Soderling, and Beavo, Curr Opin Cell Biol, vol. 12: 174-179 (2000); Francis, et al., Prog Nucleic Acid Res Mol Biol, vol. 65: 1-52 (2001); and Lerner, and Epstein, Biochem J, vol. 393: 21-41 (2006)). Some are cAMP-selective hydrolases (PDE 4, -7 and -8), others are cGMP-selective hydrolases (PDE 5, -6 and -9) and the rest hydrolyze both cAMP and cGMP (PDE1, -2, -3, -10 and -11). A review of the expression and function of the PDE family in normal haematopoietic cells is found, e.g., in Lerner, and Epstein, Biochem J, vol. 393: 21-41 (2006), the teachings of which are hereby incorporated by reference in their entirety. Additional PDEs are disclosed, for example, in U.S. Patent Application Publication No. 2005/0058998 by Beavo et al., the teachings of which are hereby incorporated by reference in their entirety.

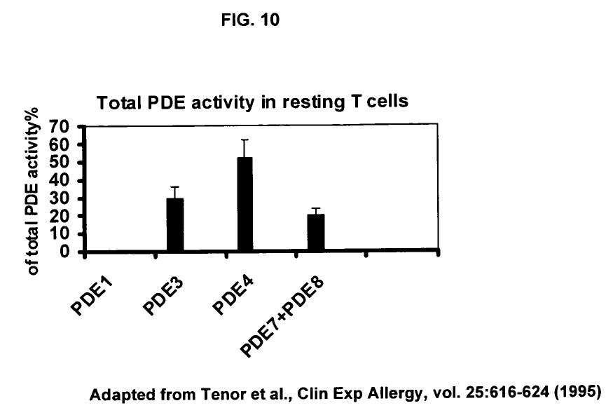

The role of PDEs in controlling the cAMP signaling with respect to T cell function is exemplified by a report by Beavo and colleagues that showed PDE7 is required for T cell activation and that PDE7 and PDE8 are induced by anti-CD3 and anti-CD28 (Li et al., Science, vol. 283:848-851 (1999); and Glavas et al., Proc Natl Acad Sci USA, vol. 98:6319-6324 (2001)). When cAMP is increased by inhibiting PDE7A there is also less IL-2 production which leads to less T cell proliferation (Li et al., Science, vol. 283:848-851 (1999)). A comprehensive review of the expression of PDEs in T cells has recently been published (Lerner and Epstein. Biochem J, vol. 393:21-41 (2006)). Initial studies have shown the predominant activity in the cytosol of isolated human peripheral blood lymphocytes to be comprised of high affinity cAMP-specific activity with kinetic, catalytic and inhibitor specificity properties, molecular mass and elution profile from anion exchange columns to be representative of PDE4 (Epstein, et al., Adv Cyclic Nucleotide Protein Phosphorylation Res, vol. 16:303-324 (1984)). Later, after the PDE1-5 gene families were discovered and defined, expression pattern of PDEs in highly purified populations of CD4+ and CD8+ human T cells were examined by activity analysis using family-specific PDE inhibitors to assess the relative contributions of the different PDE families known to exist at that time (FIG. 10) (Tenor, et al., Clin Exp Allergy, vol. 25:616-624 (1995)). Both CD4+ and CD8+ T cells showed identical PDE expression patterns, with PDE4 comprising about 60% of the total activity and PDE3 about 25%. The majority of the PDE4 activity was cytosolic, whereas the PDE3 activity was almost exclusively particulate. Only very low amounts (≦1-3%) of PDE1, PDE2 and PDE5 were expressed, and about 15% of the total activity could not be assigned to PDEs 1-5, owing to its insensitivity to specific inhibitors of these families (Tenor, et al., Clin Exp Allergy, vol. 25:616-624 (1995)). Inasmuch as the residual 15% unassigned activity exhibited a very low Km of ≈0.05-0.08 μM, it was theorized that this might represent the then newly discovered PDE7, which possesses a Km for cAMP in that range. Indeed, the PDE7A gene was subsequently shown to be expressed in human T lymphocyte cell lines (Bloom and Beavo, Proc Natl Acad Sci USA, vol. 93:14188-14192 (1996)) and in isolated human peripheral blood T lymphocytes (Giembycz, et al., Br J Pharmacol, vol. 118:1945-1958 (1996)).

Analyses of the expression patterns of PDEs in mice have not been studied as extensively as that in humans, but where reported, the expression patterns of PDEs in mice appear similar to that in humans. A single report on PDEs in isolated mouse thymocytes was published in which the PDE activity in the cytosol of these cells, as analyzed by inhibitor sensitivities, was shown to consist of about 80% PDE4 and about 20% PDE2 (Michie, et al. Cell Signal, vol. 8:97-110 (1996)). Shortly afterward the PDE7A gene was cloned from mouse and it was shown to be 98% identical to the human PDE7A gene, and the mouse PDE7A1 splice variant was shown to exhibit an identical tissue expression pattern to that in human, with highest expression in tissues of the immune system—thymus, spleen and lymph nodes (Wang, et al., Biochem Biophys Res Commun, vol. 276:1271-1277 (2000)). A subsequent study on a PDE7A gene knockout in mouse showed measurable PDE3, PDE4 and PDE7 activities in whole cell lysates of lymphocytes isolated from mouse lymph nodes, with activity levels of each of these PDEs similar to those seen in humans (Yang, et al., J Immunol, vol. 171:6414-6420 (2003)). Thus the PDE expression patterns in resting human and mouse T lymphocytes appear to be nearly identical, with the possible exception of more PDE2 expressed in mouse than in human, at least based on one report.

In addition to the expression of PDE3, PDE4 and PDE7 in resting lymphocytes, members of the PDE1 and PDE8 gene families are found in activated cells. Whereas resting lymphocytes contain little or no detectable PDE1, appreciable expression of the PDE1B gene is seen in human leukemic cells (Epstein, et al., Biochem J, vol. 243:533-539 (1987)) and mouse S49 lymphoma cells (Repaske, et al., J Biol Chem, vol. 267:18683-18688 (1992)), and its expression is induced in isolated human peripheral blood lymphocytes following mitogenic stimulation (Jiang, et al., Proc Natl Acad Sci USA, vol. 93:11236-11241 (1996)). Further, cloning of the human form of PDE1B showed it to be 96% identical to that from mouse (Jiang, et al., Proc Natl Acad Sci USA, vol. 93:11236-11241 (1996)). Following the more recent discovery of the PDE8 gene family, the PDE8A gene was also shown to be induced in both human (Glavas et al., Proc Natl Acad Sci USA, vol. 98:6319-6324 (2001) and mouse (Dong, et al., Biochem Biophys Res Commun, vol. 345:713-719 (2006)) T cells following mitogenic stimulation. Two splice variants of the PDE7A gene, 7A1 and 7A3 are also induced following activation of human T cells (Li et al., Science, vol. 283:848-851 (1999); and Glavas et al., Proc Natl Acad Sci USA, vol. 98:6319-6324 (2001)). Activation of lymphocytes also results in induction of members of the PDE4 gene family, PDE4A4, PDE4D1/D2, and PDE4D3 (Jiang, et al., Cell Biochem Biophys, vol. 28:135-160 (1998)), as well as translocation of PDE4A4, PDE4B2 and PDED1/D2 to lipid rafts (Abrahamsen et al., J Immunol, vol. 173:4847-4858 (2004)). Of the three known PDE1 genes, T cells express the PDE1B gene (Jiang, et al., Proc Natl Acad Sci USA, vol. 93:11236-11241 (1996)); of the two known PDE3 genes they express the PDE3B gene (Sheth, et al. Br J Haematol, vol. 99:784-789 (1997); and Seybold, et al., J Biol Chem, vol. 273:20575-20588 (1998)); of the four known PDE4 genes they express the PDE4A, 4B and 4D genes (Giembycz, et al., Br J Pharmacol, vol. 118:1945-1958 (1996); Jiang, et al., Cell Biochem Biophys, vol. 28:135-160 (1998); and Seybold, et al., J Biol Chem, vol. 273:20575-20588 (1998)); of the two known PDE7 genes they express the PDE7A gene (Giembycz, et al., Br J Pharmacol, vol. 118:1945-1958 (1996); and Yang, et al., J Immunol, vol. 171:6414-6420 (2003)); and of the two known PDE8 genes, they express the PDE8A gene (Glavas et al., Proc Natl Acad Sci USA, vol. 98:6319-6324 (2001)).

### Targeting of Mouse Lymphocyte PDEs as a Model for Targeting of Human T Cell PDEs in the Treatment of Human Autoimmune Diseases

Mouse models employing specific PDE gene knockouts represent excellent models for targeting these PDEs in human diseases because 1) PDEs are encoded by 21 different genes in humans, and mice express these same 21 PDE genes 2) all 21 PDE genes contain a catalytic domain of about 270 amino acids near their carboxyl terminal end which exhibits 20-45% sequence homology among all of these expressed gene products; however, for any given expressed PDE gene, the sequence homology is >95% between mouse and human species, and 3) as a result of this close sequence identity, family specific PDE inhibitors that target human forms of PDE show the exact same potency and specificity for the corresponding mouse PDEs (Bender and Beavo, Pharmacol Rev, vol. 58:488-520 (2006); Francis, et al., Prog Nucleic Acid Res Mol Biol, vol. 65:1-52 (2001); and Conti and Jin, Prog Nucleic Acid Res Mol Biol, vol. 63:1-38 (1999)).

PDE Control of T Cell Functions During Inflammation in Mutant Mice and Mice Treated with Selective Inhibitors

Over the last 15 years there has been considerable interest in the cAMP-specific, or PDE4, family of enzymes as intracellular targets that could be exploited to therapeutic advantage for a multitude of diseases associated with chronic inflammation (Burnouf and Pruniaux, Curr Pharm Des, vol. 8:1255-1296 (2002); Castro, et al., Med Res Rev, vol. 25:229-244 (2005); Barnette, et al., J Pharmacol Exp Ther, vol. 284:420-426 (1998); and Souness, et al., Immunopharmacology, vol. 47:127-162 (2000)). However, although drug targeting of PDE4 is based on a conceptually robust hypothesis (Giembycz, Proc Am Thorac Soc, vol. 2:326-333; discussion 340-321 (2005); Giembycz, Curr Opin Pharmacol, vol. 5:238-244 (2005); Bender and Beavo, Pharmacol Rev, vol. 58:488-520 (2006); Bernareggi, et al., Br J Pharmacol, vol. 128:327-336 (1999); Giembycz, Br J Clin Pharmacol, vol. 62:138-152 (2006); Sommer et al., J Neuroimmunol, vol. 79:54-61. (1997); Jung et al., J Neuroimmunol, vol. 68:1-11 (1996); Moore et al., J Pharmacol Exp Ther, vol. 319:63-72 (2006); Martinez et al., Brain Res, vol. 846:265-267 (1999); Bielekova, et al., J Immunol, vol. 164:1117-1124 (2000); Lagente et al., Mem Inst Oswaldo Cruz, vol. 100 Suppl 1:131-136 (2005); and Ouagued et al., Pulm Pharmacol Ther, vol. 18:49-54 (2005)), dose-limiting side effects, of which nausea and vomiting are the most common and troublesome, have hampered their clinical development. A fundamental challenge that still is to be met by the pharmaceutical industry is to synthesize compounds with an improved therapeutic ratio given that the adverse effects of PDE4 inhibitors represent an extension of their pharmacology. Several strategies are being considered to dissociate the beneficial from detrimental effects of PDE4 inhibitors with some degree of success (Giembycz, Proc Am Thorac Soc, vol. 2:326-333; discussion 340-321 (2005); Giembycz, Curr Opin Pharmacol, vol. 5:238-244 (2005); Bender and Beavo. Pharmacol Rev, vol. 58:488-520 (2006); Bernareggi, et al., Br J Pharmacol, vol. 128:327-336 (1999); and Giembycz, Br J Clin Pharmacol, vol. 62:138-152 (2006)). However, compounds with an optimal pharmacophore have not yet been reported. An alternative approach, that is a subject of current research, is to inhibit other cAMP PDE families that are expressed in immune and proinflammatory cells in the hope that therapeutic activity can be retained at the expense of side effects. The most promising candidates are PDE7 and PDE8. This study was carried out to assess the contribution of each of the seven PDE genes found to be expressed in T cells, PDEs 1B, 3B, 4A, 4B, 4D, 7A, and 8A, for their role in regulation of inflammatory T cells functions, and as targets for the amelioration of the clinical signs of experimental inflammation in vivo.

PDE4 is one of the major PDE gene families expressed in both human (Epstein, and Hachisu, Adv Cyclic Nucleotide Protein Phosphorylation Res, vol. 16: 303-324 (1984); Tenor, et al., Clin Exp Allergy, vol. 25: 616-624 (1995); and Giembycz, et al., Br J Pharmacol, vol. 118: 1945-1958 (1996)) and mouse (Michie, et al., Cell Signal, vol. 8: 97-110 (1996)) lymphocytes, accounting for most of the hydrolysis of cAMP. With this observation in mind, the PDE4-selective inhibitor, rolipram, was tested in previous studies to determine its effect on lymphocyte chemotaxis. This PDE4-selective inhibitor was found to inhibit lymphocyte migration stimulated by platelet activating factor, interleukin-8, and CXCL12 (stromal cell-derived factor-1) (Hidi, et al., Eur Respir J, vol. 15: 342-349 (2000); and Layseca-Espinosa, et al., J Invest Dermatol, vol. 121: 81-87 (2003)).

These in vitro studies showing effects of rolipram on lymphocyte migration were done with unstimulated, quiescent lymphocytes, but it is widely accepted that the population of lymphocytes that migrate to the site of inflammation and across the endothelium in vivo mostly belong to previously activated lymphocyte subsets and represent activated cells with a T helper type I phenotype (Nourshargh, and Marelli-Berg, Trends Immunol, vol. 26: 157-165 (2005)). Moreover, it is also widely known that the expression profile and localizations of PDEs in activated lymphocytes differ from that in unstimulated cells. Indeed, early studies had shown a long term induction of 5-10-fold in lymphocyte PDE activity following stimulation by phytohaemagglutinin (Epstein, et al., Cancer Res, vol. 40: 379-386 (1980)) or concanavalin A (Con A) (Epstein, et al., Cancer Res, vol. 40: 379-386 (1980); and Takemoto, et al., Biochem Biophys Res Commun, vol. 90: 491-497 (1979)). In addition, subsequent studies reported the long term induction in activated lymphocytes of a variety of specific PDE forms, such as PDE1B1 (Jiang, et al., Proc Natl Acad Sci USA, vol. 93 11236-11241 (1996)), PDE1B2 (Bender, et al., Proc Natl Acad Sci USA, vol. 102: 497-502 (2005)), PDE4A4 (Jiang, et al., Cell Biochem Biophys, vol. 28: 135-160 (1998)), PDE4D1/D2 (Jiang, et al., Cell Biochem Biophys, vol. 28: 135-160 (1998)), PDE4D3 (Jiang, et al., Cell Biochem Biophys, vol. 28: 135-160 (1998)), PDE7A1 (Li, et al., Science, vol. 283: 848-851 (1999)), PDE7A3 (Glavas, et al., Proc Natl Acad Sci USA, vol. 98 6319-6324 (2001)) and PDE8A1 (Glavas, et al., Proc Natl Acad Sci USA, vol. 98 6319-6324 (2001)). Furthermore, studies have also reported the translocation of PDE4A4, PDE4B2 and PDE4D1/D2 to lipid rafts following lymphocyte activation (Abrahamsen, et al., J Immunol, vol. 173: 4847-4858 (2004)).

Given these changes in PDEs that occur following lymphocyte activation, it was hypothesized that the modulation of chemotactic responses of activated lymphocytes by agents that stimulate the cAMP signaling pathway could be quite different from that of unstimulated lymphocytes. The studies disclosed herein were carried out to examine this hypothesis. The modulation of the chemotactic response exhibited by unstimulated and Con A-stimulated splenocytes in the presence of agents that stimulate the cAMP signaling pathway was compared. Whereas the cell permeable cAMP analogue, dibutyryl cAMP readily inhibited chemotaxis of both cell populations, surprisingly, the adenylyl cyclase activator, forskolin, and the general non-selective PDE inhibitor, IBMX, inhibited migration of unstimulated splenocytes, but either had no effect at all (forskolin) or only a limited effect (IBMX) on activated cells, except when added together. Since direct addition of an analogue of cAMP inhibits migration of both cell types, whereas broad modulators of the synthetic and degradative enzymes that regulate cAMP primarily inhibited migration only of unstimulated cells, unstimulated and activated splenocytes must differ in the way in which they regulate cAMP within the cell.

Stimulated splenocytes, like unstimulated splenocytes, responded to the chemokine CXCL12, a powerful chemoattractant for leukocytes (Rot and von Andrian, Annu Rev Immunol, vol. 22: 891-928 (2004)), and stimulated splenocytes were readily inhibited in their migration by the cAMP analogue, dibutyryl cAMP; however, unlike unstimulated cells, migration of stimulated splenocytes was not significantly affected by activators of adenylyl cyclase or inhibitors of cAMP PDEs, except for dipyridamole, which differs from the other PDE inhibitors used only in its ability to inhibit PDE8. Further, quantitative real-time RTPCR revealed an induction of PDE8 mRNA in splenocytes following Con A stimulation. The studies disclosed herein demonstrate that, for activated lymphocytes, the major therapeutic target in inflammatory autoimmune diseases, inhibition of PDE4 is not sufficient to block their recruitment into sites of inflammation. Thus, the methods provided herein inhibit PDE8 to achieve a full therapeutic response.

It has become increasingly apparent in recent years that in response to stimuli, cAMP elevations in cells occur in a directed spatial and temporal manner, resulting in the formation of microdomains of localized cAMP concentrations within the cell, and it is the regulation of these localized domains of cAMP in physiologically important compartments that regulate specific functions of the cell (Rich, et al., Proc Natl Acad Sci USA, vol. 98: 13049-13054 (2001)). It is now established that one way microdomains of localized cAMP concentrations are achieved in the cell is through selective expression and compartmentalization of different isoforms of PDE (Rich, et al., J Gen Physiol, vol. 118: 63-78 (2001); Brunton, Sci STKE 2003: PE44 (2003); Mongillo, et al., Circ Res, vol. 95: 67-75 (2004); and Baillie, et al., FEBS Lett, vol. 579: 3264-3270 (2005)). Since migration of unstimulated splenocytes was readily inhibited by the general non-selective PDE inhibitor, IBMX, and activated splenocytes were far less affected by IBMX, it was hypothesized that activated splenocytes might express an IBMX-insensitive PDE activity in a functionally relevant cell compartment linked to regulation of cell migration, which limits the accumulation of cAMP in that compartment in response to activators of adenylyl cyclase, IBMX or PDE selective inhibitors targeted to IBMX-sensitive PDE gene families. Indeed, in addition to the relative insensitivity to IBMX, migration of activated splenocytes was also found to be resistant to inhibition by gene family specific inhibitors targeted to PDE3, PDE4, and PDE7. The inhibition of stimulated splenocytes seen following combined addition of forskolin and IBMX could result from excessive increases in cAMP causing spillover from one cellular compartment to another, and overwhelming the normal cAMP degradative system in the compartment responsible for regulation of migration.

Inasmuch as PDE8 is the only known cAMP hydrolyzing PDE gene family reported to be resistant to IBMX inhibition, studies were carried out to evaluate whether high expression of PDE8 in activated splenocytes confers resistance of these cells to inhibition of migration by adenylyl cyclase activators and PDE inhibitors. If true, then inhibition of PDE8 in activated splenocytes should inhibit their chemotactic migration. Pharmacological characterization of expressed forms of PDE8 showed them to be resistant to inhibition by all known PDE inhibitors tested against them, with the exception of the non-selective PDE inhibitor, dipyridamole, which inhibits PDE8A with reported IC50s of 4-9 μM (Soderling, et al., Proc Natl Acad Sci USA, vol. 95: 8991-8996 (1998); Fisher, et al., Biochem Biophys Res Commun, vol. 246: 570-577 (1998); and Gamanuma, et al., Cell Signal, vol. 15: 565-574 (2003)).

The structure of dipyridamole is as follows:

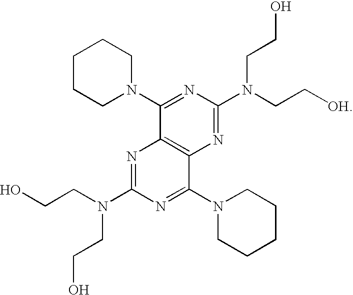

Dipyridamole was then tested in the chemotactic assay system described in the Examples below. In support of the hypothesis, the studies disclosed herein demonstrated that dipyridamole profoundly inhibited chemotactic migration of both unstimulated and stimulated splenocytes.

Moreover, when forskolin was added along with dipyridamole, to stimulate adenylyl cyclase, inhibition of migration was potentiated, leading to as much as =70-80% inhibition of migration of both unstimulated and stimulated cells. The structure of forskolin is as follows:

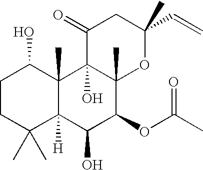

That dipyridamole is working through a cAMP mediated effect is borne out by the reversal of the dipyridamole effect that is seen with the protein kinase A (PKA) antagonist, Rp-cAMPS.

In a further test of the hypothesis, quantitative real-time RT-PCR was performed to assess the mRNA expression for PDE4B2, PDE7A1, and PDE8A1, following Con A stimulation of splenocytes. Consistent with the hypothesis, a 2.7-fold induction of mRNA for PDE8A1 was found, with no increase in mRNA for PDE 7A1, and only a transient 1.5-fold increase in mRNA for PDE4B2. Hence, it is conceivable that this increase in the expression of PDE8A in activated splenocytes is responsible for conferring relative resistance of these cells to inhibition of chemotactic migration by adenylyl cyclase activators and PDE inhibitors other than dipyridamole.

Studies have been run to determine why lymphocytes induce PDE8 in a cell compartment functionally linked to regulation of migration following cell activation. Nearly 30 years ago studies reported 10-20-fold increases in PDE activity in proliferating and transformed lymphocytes as compared to quiescent cells, and a 5-10-fold, long term induction of PDE activity in human peripheral blood lymphocytes following mitogenic stimulation. Based on these findings, it was postulated that induction of PDE might be important to the mitogenic process (Epstein, et al., Cancer Res, vol. 40: 379-386 (1980); and Epstein, et al., Cancer Res, vol. 37: 4016-4023 (1977)). It was then hypothesized that the increased PDE activity might serve as a protective mechanism to ensure that cAMP levels are not elevated, from circulating hormones and other activators of adenylyl cyclase such as adenosine, to the point of being inhibitory to activated lymphocytes, thereby ensuring that the cells can traverse the stages necessary for commitment to mitogenesis. Subsequent studies have largely borne this out and have shown that PDEs 1 and 4, both of which are induced, are to a large extent, responsible for controlling cAMP levels linked to proliferation following lymphocyte activation (Jiang, et al., Proc Natl Acad Sci USA, vol. 93 11236-11241 (1996); Jiang, et al., Cell Biochem Biophys, vol. 28: 135-160 (1998); and Kanda, and Watanabe, Biochem Pharmacol, vol. 62: 495-507 (2001)). A similar hypothesis is tested herein; that is, that the induction of PDE8, in part, functions to protect the activated lymphocytes from being inhibited in their migration by agents in the circulation that can stimulate adenylyl cyclase, and elevate cAMP in a compartment that can affect migration.

Previous studies had reported induction of PDE7A1 in human T lymphocytes activated with anti-CD3/CD28 antibodies, and the inhibition of T cell proliferation by antisense inhibition of PDE7A1 expression (Li, et al., Science, vol. 283: 848-851 (1999)). It was therefore surprising to see no change in PDE7A1 mRNA in mouse splenocytes following their activation. However, the complement of PDEs in human and mouse T lymphocytes differ. For example, whereas mice express PDE2 as one of the major PDE forms in T cells (Michie, et al., Cell Signal, vol. 8: 97-110 (1996)), PDE2 is not expressed at all in human T or B cells (Tenor, et al., Clin Exp Allergy, vol. 25: 616-624 (1995) and Gantner, et al., Br J Pharmacol, vol. 123: 1031-1038 (1998)). Further, T lymphocyte activation and cytokine production were completely normal in PDE7A knockout mice, suggesting that in contrast to humans, PDE7A may have little or no functional role in murine T cells (Yang, et al. J Immunol, vol. 171: 6414-6420 (2003)). In contrast, similar to what is shown in the Examples below in mice, PDE8A mRNA was upregulated following activation of human T cells, and was hypothesized to play an important role in the activation process, suggesting that PDE8 plays similar functionally important roles in both human and murine lymphocytes (Glavas, et al., Proc Natl Acad Sci USA, vol. 98 6319-6324 (2001)).

Dipyridamole has other actions in addition to its inhibition of PDEs, and it is possible that its inhibition of migration of activated splenocytes may be due to another action. Dipyridamole is a potent inhibitor of adenosine uptake into cells, and is used clinically, under the trade name Persantine, for this action during pharmacological stress tests. Since adenosine deaminase had no effect on migration of splenocytes and did not attenuate the dipyridamole inhibition of migration, it suggests that dipyridamole is not acting in this system through inhibition of adenosine uptake. However, even though dipyridamole may act to elevate extracellular adenosine, the inhibitory actions of extracellular adenosine on lymphocyte proliferation and function are nevertheless believed to result from elevation of intracellular cAMP, as a result of stimulation of adenylyl cyclase through purinergic receptors (DosReis, et al., Cell Immunol, vol. 101: 213-231 (1986)). Hence, dipyridamole is thought to stimulate the cAMP signaling pathway in two ways, through indirect stimulation of adenylyl cyclase as a result of elevation of adenosine, and through direct inhibition of PDEs. Dipyridamole has anti-inflammatory properties and was recently reported to attenuate nuclear translocation of NFκB and block the synthesis of monocyte chemoattractant protein-1 in platelet-monocyte aggregates (Weyrich, et al., Circulation, vol. 111: 633-642 (2005)). Much of the anti-inflammatory properties of dipyridamole results from its ability to inhibit chemotactic migration of activated lymphocytes, as shown in the study provided herein.

During inflammation, leukocytes respond to chemokines presented at the luminal surface of the endothelium through their G-protein coupled high affinity receptors and migrate into underlying tissues. Since chemokine-induced transendothelial migration of activated T lymphocytes is one of the first steps of the pathological process in chronic inflammatory autoimmune diseases such as multiple sclerosis, blockage of this migration should provide an effective therapeutic means of treating autoimmune diseases (Steinman, Science 305: 212-216 (2004) and Steinman, Nat Rev Drug Discov, vol. 4: 510-518 (2005)). A review of the pathophysiology of autoimmune diseases and targeted immune therapies for autoimmune diseases is provided, e.g., in Steinman, Science 305: 212-216 (2004), the teachings of which are hereby incorporated by reference in their entirety.

The studies provided herein show that PDE8 is a critical target for inhibiting the migration of activated lymphocytes. The methods of modulating, e.g., treating, reducing, alleviating or otherwise preventing, inflammation or other immune-related diseases employ selective PDE inhibitors, and preferably, at least a selective PDE8 inhibitor. The selective PDE8 inhibitor is, for example, a commercially available selective inhibitor, such as dipyridamole. Other suitable selective PDE8 inhibitors include compounds identified by screening known chemical libraries for novel compounds that inhibit PDE8 with the same or better ability as dipyridamole. Other suitable selective PDE8 inhibitors are created using strategies such as rational design, for example, based on the structure of dipyridamole or a derivative thereof. Putative selective PDE8 inhibitors are screened and/or tested for their ability to inhibit the migration of stimulated and unstimulated splenocytes, for example, using the chemotaxis assays disclosed herein.

All mammalian PDEs identified to date contain a catalytic region comprised of approximately 270 amino acids located toward the C-terminus. The different PDEs share between about 20-45% identity in this region. Crystal structures have been determined for the catalytic sites of PDEs 1, 3, 4, 5, 7 and 9, but not yet for PDE8. Development of PDE8 inhibitors is based on the principles of binding of substrate and inhibitors to the catalytic site derived from these solved crystal structures. In particular, since PDE8 and PDE4 are strictly cAMP-specific, information gathered from binding to the active site of PDE4 is used in the development of PDE8 inhibitors.

From all the PDE crystal structures solved so far, the catalytic domains are composed of 16 alpha helices consisting of three sub-domains that define a deep pocket where substrate or inhibitors bind. The active site pocket contains 11 of the 17 conserved residues in all PDEs. Important residues are contributed by each of the 3 sub-domains. Two divalent metal binding sites, both involved in catalysis, are found at the bottom of the substrate binding pocket; one site for a tightly bound Zn++ and another for a loosely bound Mg++. The Zn++ binding site has two histidine and two aspartic acid residues that are conserved among all known PDEs. All structures show an invariant glutamine that stabilizes the binding of the purine ring. For dual specificity PDEs, this glutamine must be able to rotate freely to form hydrogen bonds with both cAMP and cGMP. For PDEs that are specific for cAMP, this glutamine is constrained by neighboring residues into a favored orientation for cAMP, and similarly, for cGMP-specific PDEs, it is constrained into a different position that favors cGMP. As PDE8 and PDE4 are both cAMP-specific, inhibitors of PDE8 are based on structural determinants of dipyridamole, E4021 and papaverine, which have some ability to inhibit PDE8, and the known residues important for substrate and inhibitor binding of PDE4.

The crystal structure of the catalytic domain of PDE4B, the first to be solved, and a model for the binding of cAMP substrate to PDE4B was proposed. The preferred model for cAMP binding to the catalytic pocket of PDE4B is the anti conformation of cAMP where the adenine base is inserted into the lipophilic pocket by Leu393, Pro396, Ile410, Phe414, and Phe446. The cyclic phosphate group binds to the two metal binding sites. The 1-N and 6-NH₂ groups form hydrogen bonds with the side chain of the invariant Gln443, while the 7-N position forms a more distorted hydrogen bond with asn395. The ribose ring binds loosely against Met347 and Leu393, with a hydrogen bond between His234 and the O3′ oxygen, but with no obvious interaction with the O2′, O4′, and O5′ atoms.

Interaction of PDEs with scaffold proteins, which target them to specific subcellular regions, is critical for correct signaling to occur. Hence, interference of the specific binding of PDEs to their scaffold proteins disrupts their function. PDE8 is the only PDE gene family that contains a PAS (Per-Arnt-Sim) domain through which it most likely binds to other proteins. Indeed, a PAS-dependent physical association of PDE8A1 with endogenous IkappaB has been shown by antibody array. Binding of PDE8A1 competes with the p65/p50 NF-kappaB for IkappaB binding, and the binding of IkappaB to PDE8A increases its catalytic activity 6-fold. Therefore, inhibitors are designed to target to the PAS region of PDE8A to disrupt the binding of PDE8A to other partner or scaffold proteins, as a means to further block the function of this enzyme.

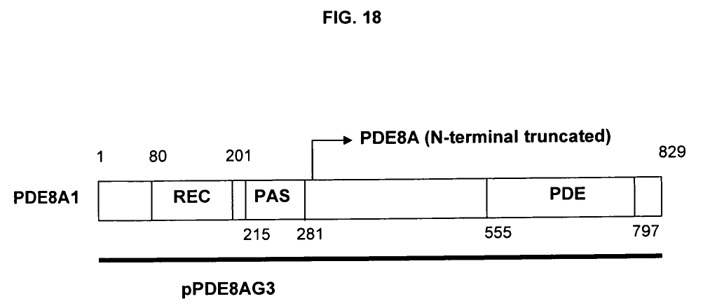

The PDE8 inhibitors are designed to bind to at least a portion the catalytic domain of the PDE8A1 isoform so as the interfere with the interaction between PDE8A1 and cAMP. Preferably, these PDE8 inhibitor competes with cAMP for binding to the active site of PDE8A1. A diagram of the PDE8A1 protein is shown in FIG. 18. These PDE8 inhibitors are designed to bind to the catalytic domain region of PDE8A1, which is located between amino acid residues 555-797 of the amino acid sequence shown in GenBank Accession No. NP_{—}002596, or the corresponding amino acid residues encoded by the PDE8A1 nucleic acid sequences in GenBank Accession Nos. NM_{—}002605, BC060762; BC075822 and NM173454.

Other suitable PDE8 inhibitors are designed to bind to a portion of the PDE8A1 isoform that is separate and distinct from the catalytic domain, wherein that portion of PDE8A1 is a binding site for a secondary molecule, such as a chaperone protein. For example, PDE8 inhibitors are designed to bind to at least a portion of the PAS region of PDE8A1 so as the interfere with the interaction between PDE8A1 and a chaperone protein, such as, for example, I kappa beta. Preferably, these PDE8 inhibitor competes with I kappa β for binding to the PAS region of PDE8A1. These PDE8 inhibitors are designed to bind to the PAS region of PDE8A1, which is located between amino acid residues 215-281 of the amino acid sequence shown in GenBank Accession No. NP_{—}002596, or the corresponding amino acid residues encoded by the PDE8A1 nucleic acid sequences in GenBank Accession Nos. NM_{—}002605, BC060762; BC075822 and NM173454.

Selective inhibitors of PDE8 are administered alone or in combination with other suitable therapeutic agents. For example, the selective PDE8 inhibitor is administered in combination with one or more additional PDE inhibitors, such as, a PDE4 inhibitor (Bielekova, et al., J Immunol, vol. 164:1117-1124 (2000)), a PDE7 inhibitor (Li et al., Science 283:848-851 (1999); and Glavas et al., Proc Natl Acad Sci USA 98:6319-6324 (2001)) or both a PDE4 inhibitor and a PDE7 inhibitor.

The selective PDE inhibitors used in the methods of the invention, such as, the selective PDE8 inhibitors, are administered in an amount that is effective to treat, reduce, alleviate or otherwise prevent multiple sclerosis and other autoimmune diseases associated with chemokine-induced migration of leukocytes. For example, the selective PDE inhibitors are used in an amount that is effective to treat, reduce, alleviate, delay the progression of or otherwise prevent an autoimmune disease or allergic disease selected from the group comprising multiple sclerosis, type 1 diabetes, rheumatoid arthritis, asthma, chronic obstructive pulmonary diseases, inflammatory bowel disease, Alzheimer's disease and other neurodegenerative diseases with inflammatory components, atherosclerosis, vasculitis, and cancer.

As used herein, the term “antagonist” or “inhibitor” refers to a molecule which, when bound to a PDE, decreases the amount (i.e., expression) or the duration of the effect of the biological or immunological activity of the PDE. Antagonists may include small molecules, proteins, polypeptides, peptides, nucleic acids, carbohydrates, antibodies or any other molecules which decrease the amount (expression) or effect of PDEs present in the sample. The preferred antagonist selectively inhibits the biological activity of a PDE, while not affecting any other cellular proteins.

As used herein, the term “modulates” refers to a change in the activity of PDEs. For example, modulation may cause an increase or a decrease in protein amount or activity, binding characteristics, or any other biological, functional or immunological properties of PDEs.

As used herein, the term “biological sample” is used in its broadest sense. A biological sample is suspected of containing nucleic acid encoding PDEs, or fragments thereof, or a PDE protein itself or fragments thereof. The suitable biological sample is from, e.g., an animal or a human. The sample is a cell sample or a tissue sample, including samples from spleen, lymph node, thymus, bone marrow, liver, heart, testis, brain, placenta, lung, skeletal muscle, kidney and pancreas. The sample is a biological fluid, including, urine, blood sera, blood plasma, phlegm, or lavage fluid. Alternatively, the sample is a swab from the nose, ear or throat.

The term “T cell activation” as used herein refers to a process by which T cells change from a resting state to one where they are proliferating and producing interleukins. In vivo, T cell activation occurs when an antigen-presenting cell (APC) binds to the T cell via the T cell receptor/CD3 complex and another co-stimulatory molecule, such as CD28. In vitro, T cell induction can be induced by binding anti-mouse antibodies beads to a plate. When antibodies to murine anti-CD3 and anti-CD28 antibodies are added to the plate, they bind to the anti-mouse antibodies by their Fc regions. This leaves the Fab region free to bind CD3 and CD28 receptors on T cells. When T cells are added to the plate, they bind to the antibodies attached to the bottom of the plate and become activated, resulting in T cell proliferation and production of interleukins. The plate with attached antibodies approximates an APC which has receptors that bind to CD3 and CD28.

A variety of abbreviations are used throughout. “cAMP” is an abbreviation for cyclic AMP or cyclic 3′,5′-adenosine monophosphate. cAMP is a small nucleotide regulatory molecule that functions to regulate many fundamental cell processes. “cGMP” is cyclic 3′,5′-guanosine monophosphate, another regulatory molecule that can be hydrolyzed by some forms of PDE. “COPD” refers to chronic obstructive pulmonary disease. “EAE” refers to experimental autoimmune encephalomyelitis, a T cell-mediated inflammatory disease of the CNS that serves as an animal model of the human disease multiple sclerosis. “GAP” refers to GTPase activating protein which accelerates the intrinsic GTP-hydrolytic activity of RhoA to produce the GDP-bound inactive state. “GEF” refers to guanine nucleotide exchange factor which promotes GDP release and GTP binding to RhoA. “GDI” refers to guanine nucleotide dissociation inhibitor which sequesters RhoA in its GDP bound state and interferes with its membrane association. “mAb” refers to monoclonal antibody. “PDE” refers to an abbreviation for cyclic nucleotide phosphodiesterase(s). These are the enzymes that terminate the actions of cAMP by breaking it down to 5′-AMP, and they are thus predominantly responsible for controlling the levels of cAMP in cells. “PKA” refers to cAMP-dependent protein kinase. “qRT-PCR” refers to quantitative real-time reverse transcriptase-polymerase chain reaction. “RhoA” refers to a small GTP binding protein that possesses GTPase activity belonging to the ras superfamily of GTP binding proteins. “ROCK” refers to a protein kinase that is activated by RhoA. “Rp-cAMPS” refers to 3′,5′-cyclic monophosphorothioate, Rp-isomer. This is a stereoisomer analogue of cAMP that acts as an antagonist of PKA by competing for cAMP for binding to the PKA regulatory subunits.

### Pharmaceutical Compositions

The compounds of the invention can be useful in the prevention or treatment of a variety of human or other animal, including mammalian and non mammalian, disorders, including primarily inflammatory disorders and other immune-related diseases. It is contemplated that, once identified, the active molecules of the invention, such as the selective PDE inhibitor, preferably at least a selective PDE8 inhibitor, can be incorporated into any suitable carrier prior to use. The dose of active molecule, mode of administration and use of suitable carrier will depend upon the intended recipient and target disorder. The formulations, both for veterinary and for human medical use, of inhibitors according to the present invention typically include such inhibitors in association with a pharmaceutically acceptable carrier.

The carrier(s) should be “acceptable” in the sense of being compatible with the other ingredients of the formulations and not deleterious to the recipient. Pharmaceutically acceptable carriers, in this regard, are intended to include any and all solvents, dispersion media, coatings, anti-bacterial and anti-fungal agents, isotonic and absorption delaying agents, and the like, compatible with pharmaceutical administration. The use of such media and agents for pharmaceutically active substances is known in the art. Except insofar as any conventional media or agent is incompatible with the active compound, use thereof in the compositions is contemplated. Supplementary active compounds (identified or designed according to the invention and/or known in the art) also can be incorporated into the compositions. The formulations can conveniently be presented in dosage unit form and can be prepared by any of the methods well known in the art of pharmacy/microbiology. In general, some formulations are prepared by bringing the inhibitors into association with a liquid carrier or a finely divided solid carrier or both, and then, if necessary, shaping the product into the desired formulation.

A pharmaceutical composition of the invention should be formulated to be compatible with its intended route of administration. Examples of routes of administration include oral or parenteral, for example, intravenous, intradermal, inhalation, transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide.

Useful solutions for oral or parenteral administration can be prepared by any of the methods well known in the pharmaceutical art, described, for example, in Remington's Pharmaceutical Sciences, (Gennaro, A., ed.), Mack Pub., (1990). The parenteral preparation can be enclosed in ampoules, disposable syringes or multiple dose vials made of glass or plastic. Suppositories for rectal administration also can be prepared by mixing the drug with a non-irritating excipient such as cocoa butter, other glycerides, or other compositions which are solid at room temperature and liquid at body temperatures. Formulations also can include, for example, polyalkylene glycols such as polyethylene glycol, oils of vegetable origin, and hydrogenated naphthalenes. Formulations for direct administration can include glycerol and other compositions of high viscosity. Other potentially useful parenteral carriers for these drugs include ethylene-vinyl acetate copolymer particles, osmotic pumps, implantable infusion systems, and liposomes. Formulations for inhalation administration can contain as excipients, for example, lactose, or can be aqueous solutions containing, for example, polyoxyethylene-9-lauryl ether, glycocholate and deoxycholate, or oily solutions for administration in the form of nasal drops, or as a gel to be applied intranasally. Retention enemas also can be used for rectal delivery.

Formulations of the present invention suitable for oral administration can be in the form of: discrete units such as capsules, gelatin capsules, sachets, tablets, troches, or lozenges, each containing a predetermined amount of the drug; a powder or granular composition; a solution or a suspension in an aqueous liquid or non-aqueous liquid; or an oil-in-water emulsion or a water-in-oil emulsion. The drug can also be administered in the form of a bolus, electuary or paste. A tablet can be made by compressing or molding the drug optionally with one or more accessory ingredients. Compressed tablets can be prepared by compressing, in a suitable machine, the drug in a free-flowing form such as a powder or granules, optionally mixed by a binder, lubricant, inert diluent, surface active or dispersing agent. Molded tablets can be made by molding, in a suitable machine, a mixture of the powdered drug and suitable carrier moistened with an inert liquid diluent.

Oral compositions generally include an inert diluent or an edible carrier. For the purpose of oral therapeutic administration, the active compound can be incorporated with excipients. Oral compositions prepared using a fluid carrier for use as a mouthwash include the compound in the fluid carrier and are applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose; a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). It should be stable under the conditions of manufacture and storage and should be preserved against the contaminating action of microorganisms such as bacteria and fungi. The carrier can be a solvent or dispersion medium containing, for example, water, ethanol, polyol (for example, glycerol, propylene glycol, and liquid polyethylene glycol), and suitable mixtures thereof. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as manitol, sorbitol, or sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filter sterilization. Generally, dispersions are prepared by incorporating the active compound into a sterile vehicle which contains a basic dispersion medium and the required other ingredients from those enumerated above. In the case of sterile powders for the preparation of sterile injectable solutions, methods of preparation include vacuum drying and freeze-drying which yields a powder of the active ingredient plus any additional desired ingredient from a previously sterile-filtered solution thereof.

Formulations suitable for intra-articular administration can be in the form of a sterile aqueous preparation of the drug that can be in microcrystalline form, for example, in the form of an aqueous microcrystalline suspension. Liposomal formulations or biodegradable polymer systems can also be used to present the drug for both intra-articular and ophthalmic administration.

Formulations suitable for topical administration, including eye treatment, include liquid or semi-liquid preparations such as liniments, lotions, gels, applicants, oil-in-water or water-in-oil emulsions such as creams, ointments or pastes; or solutions or suspensions such as drops. Formulations for topical administration to the skin surface can be prepared by dispersing the drug with a dermatologically acceptable carrier such as a lotion, cream, ointment or soap. Particularly useful are carriers capable of forming a film or layer over the skin to localize application and inhibit removal. For topical administration to internal tissue surfaces, the agent can be dispersed in a liquid tissue adhesive or other substance known to enhance adsorption to a tissue surface. For example, hydroxypropylcellulose or fibrinogen/thrombin solutions can be used to advantage. Alternatively, tissue-coating solutions, such as pectin-containing formulations can be used.

For inhalation treatments, inhalation of powder (self-propelling or spray formulations) dispensed with a spray can, a nebulizer, or an atomizer can be used. Such formulations can be in the form of a fine powder for pulmonary administration from a powder inhalation device or self-propelling powder-dispensing formulations. In the case of self-propelling solution and spray formulations, the effect can be achieved either by choice of a valve having the desired spray characteristics (i.e., being capable of producing a spray having the desired particle size) or by incorporating the active ingredient as a suspended powder in controlled particle size. For administration by inhalation, the compounds also can be delivered in the form of an aerosol spray from pressured container or dispenser which contains a suitable propellant, e.g., a gas such as carbon dioxide, or a nebulizer.

Systemic administration also can be by transmucosal or transdermal means. For transmucosal or transdermal administration, penetrants appropriate to the barrier to be permeated are used in the formulation. Such penetrants generally are known in the art, and include, for example, for transmucosal administration, detergents and bile salts. Transmucosal administration can be accomplished through the use of nasal sprays or suppositories. For transdermal administration, the active compounds typically are formulated into ointments, salves, gels, or creams as generally known in the art.

The active compounds can be prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. Liposomal suspensions can also be used as pharmaceutically acceptable carriers. These can be prepared according to methods known to those skilled in the art, for example, as described in U.S. Pat. No. 4,522,811.

Oral or parenteral compositions can be formulated in dosage unit form for ease of administration and uniformity of dosage. Dosage unit form refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals. Furthermore, administration can be by periodic injections of a bolus, or can be made more continuous by intravenous, intramuscular or intraperitoneal administration from an external reservoir (e.g., an intravenous bag).

Where adhesion to a tissue surface is desired the composition can include the drug dispersed in a fibrinogen-thrombin composition or other bioadhesive. The inhibitor then can be painted, sprayed or otherwise applied to the desired tissue surface. Alternatively, the inhibitors can be formulated for parenteral or oral administration to humans or other mammals, for example, in therapeutically effective amounts, e.g., amounts that provide appropriate concentrations of the inhibitor to target tissue for a time sufficient to induce the desired effect.

The compounds of the present invention can be administered directly to a tissue locus by applying the compound to a medical device that is placed in contact with the tissue. An example of a medical device is a stent, which contains or is coated with one or more of the compounds of the present invention.

Active compound as identified or designed by the methods described herein can be administered to individuals to treat disorders prophylactically or therapeutically. In conjunction with such treatment, pharmacogenomics (i.e., the study of the relationship between an individual's genotype and that individual's response to a foreign compound or drug) can be considered. Differences in metabolism of therapeutics can lead to severe toxicity or therapeutic failure by altering the relation between dose and blood concentration of the pharmacologically active drug. Thus, a physician or clinician can consider applying knowledge obtained in relevant pharmacogenomics studies in determining whether to administer a drug as well as tailoring the dosage and/or therapeutic regimen of treatment with the drug.

In therapeutic use for treating, or combating, inflammation and other immune-related diseases in mammals, the inhibitors or pharmaceutical compositions thereof will be administered orally, parenterally and/or topically at a dosage to obtain and maintain a concentration, that is, an amount, or blood-level or tissue level of active component in the animal undergoing treatment which will be effective to modulate inflammation, e.g., by modulating the migration of activated lymphocytes. Generally, an effective amount of dosage of active component will be in the range of from about 0.1 to about 100, more preferably from about 1.0 to about 50 mg/kg of body weight/day. The amount administered will also likely depend on such variables as the type and extent of disease or indication to be treated, the overall health status of the particular patient, the relative biological efficacy of the compound delivered, the formulation of the drug, the presence and types of excipients in the formulation, and the route of administration. Also, it is to be understood that the initial dosage administered can be increased beyond the above upper level in order to rapidly achieve the desired blood-level or tissue level, or the initial dosage can be smaller than the optimum and the daily dosage can be progressively increased during the course of treatment depending on the particular situation. If desired, the daily dose can also be divided into multiple doses for administration, for example, two to four times per day.

All cited publications, patents, patent applications, sequence information cited by GenBank, Ensembl or other public sequence database accession numbers, and all other references that are included in the attached papers and/or manuscripts are specifically incorporated by reference herein in their entirety in order to more fully describe the state of the known art pertaining to the present invention.

The invention is further described in the following examples, which do not limit the scope of the invention described in the claims.

### EXAMPLES

### Example 1

### Materials and Methods

Materials. Recombinant mouse CXCL12 was obtained from R&D Systems (Minneapolis, Minn.), forskolin and RpcAMPS from Biomol (Plymouth Meeting, Pa.), and 3-isobutyl-1-methylxanthine (IBMX), dipyridamole, Con A, dibutyryl cAMP and adenosine deaminase type X from Sigma-Aldrich (St. Louis, Mo.). The PDE3 inhibitor motapizone, the PDE4 inhibitor piclamilast and a PDE7-selective inhibitor were supplied by colleagues.

Isolation of murine splenocytes. Splenocytes were isolated from 6-8 week old C57BL/6 mice obtained from Jackson Laboratories (Bar Harbor, Me.). Spleens were removed and a single-cell suspension prepared using 40 μm cell strainers (Fisher Scientific). Cells were washed with RPMI 1640 medium supplemented with 5% fetal bovine serum, 2 mM L-glutamine, 100 U/ml penicillin and 100 mg/ml streptomycin (all from GIBCO). Red blood cells were lysed using standard lysis buffer (0.15M NH₄Cl, 10 mM KHCO₃, 0.1 mM EDTA PH 7.4). Cells were then washed and used in chemotaxis assays either as unstimulated cells or after stimulation with Con A (31 g/ml) as indicated.

Preparation of Test Reagents. Forskolin, IBMX, Motapizone, Piclamilast, and dipyridamole were dissolved as 1000× stock solutions in 100% DMSO and diluted into the chemotaxis assays to give a final DMSO concentration of 0.1%. This concentration of DMSO had no effect on migration of splenocytes in the presence or absence of CXCL12. CXCL12 was prepared as a 100 μg/ml stock solution in PBS+0.1% BSA, Con A was prepared as a stock of 2.5 mg/ml in PBS, and dibutyryl cAMP and Rp-cAMPS were prepared as 50 mM stock solutions in water, and these reagents were diluted directly into the chemotaxis assays.

Chemotaxis Assay. Chemotaxis assays were done in 24-well transwell plates with a pore size of 5 μm (Costar, Corning). Splenocytes were placed in medium at a concentration of 3×10⁶ cells/ml. Where test agents were used, splenocytes were pretreated with the agent or vehicle for 45 to 60 min, following which 100 μl of the splenocyte suspension was placed into the upper chamber of transwell plates, and the lower chamber was filled with 600 μl of medium. When added to induce migration, CXCL12 (250 ng/ml) was added to the lower chamber only. Other test reagents were added as indicated to both the upper and lower chambers. After 4 hours of incubation at 37° C. in 5% CO2, transwell inserts were gently removed and the number of cells that migrated into the lower chamber were counted by withdrawing 500 μl of lower chamber medium, mixing it with 10 ml of buffer and counting the cells on a Coulter Counter (Beckman Coulter Z series). Where results are expressed as % of CXCL12-stimulated migration, this was calculated as follows: (cells migrated in presence of CXCL12 and test reagent—cells migrated in medium alone)/(cells migrated in presence of CXCL12-cells migrated in medium alone)×100. Experimental points for all chemotaxis assays were performed in triplicate.

Quantitative real-time RT-PCR. Total RNA was isolated from unstimulated splenocytes and from splenocytes stimulated by Con A for different lengths of time as indicated, using RNeasy mini kits (Qiagen) according to the manufacturer's instructions. cDNA was synthesized using M-MLV reverse transcriptase (Promega). Primers were designed using ABI Primer Express Software v3.0. and synthesized by Invitrogen Life Technologies. Quantitative real-time RT-PCR 7 was performed using an ABI 7500 fast system and data analyzed using 7500 fast system SDS software v3.0. Sets of primers with the following sequences were used: PDE4B2 primer sequences, forward: ACCTGAGCAACCCCACCAA (SEQ ID NO:1), reverse: CCCCTCTCCCGTTCTTTGTC (SEQ ID NO:2); PDE7A primer sequences, forward: TCAGCAGCAATCTTGATGCAA (SEQ ID NO:3), reverse: AGAGGCTGGGCACTTCACAT (SEQ ID NO:4); PDE8A primer sequences, forward: CCTGCAGCATTCCCAAGTC (SEQ ID NO:5), reverse: TGTATAAGGTTAGGCAGGTCAA (SEQ ID NO:6); ribosomal protein L19 (RPL19) primer sequences, forward: CCAAGAAGATTGACCGCCAT (SEQ ID NO:7), reverse: CAGCTTGTGGATGTGCTCCAT (SEQ ID NO:8). Amplicon sizes were 100 bp.

Statistics. Data are plotted as the means±S.D. of replicate determinations. Statistical significance of experimental conditions relative to control were analyzed by Student's t-test and significance indicated by asterisks in the figure, with the p values given in the legend.

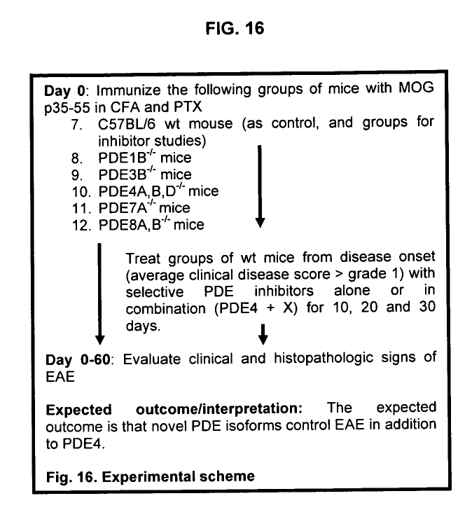

Proliferation assays. Lymphocytes and splenocytes are obtained from naive mice by separation of draining lymph nodes or spleen using cell strainers. Cells are washed twice in serum-free AIM-V culture medium and resuspended in AIM-V culture media supplemented with 10⁻⁵ M 2-ME. For proliferation assays, splenocytes (3×10⁵ cells/well) and lymph node cells (2.5×10⁵ cells/well) are plated in triplicate cultures in 96-well microtiter culture plates. Anti-CD3 and anti-CD28 mAb are immobilized according to standard procedures. Culture plates are incubated for 48 h at 37° C. Proliferation is assessed by measuring [³H]-thymidine incorporation added at 0.2 μCi/well for the last 16 h. At the end of the incubation period, cells are harvested and radioisotope incorporation measured as an index of lymphocyte proliferation in a betaplate liquid scintillation counter (FIGS. 16A, B).

In FIGS. 16A and 16B, T cells of regional draining lymph nodes from wild-type (CD26^{+/+}) and CD26^{-/-} mice were isolated on day 11 or 12 following immunization with MOG p35-55. Cells were cultured with the indicated concentrations of MOG p35-55 (FIG. 16A) or PWM (FIG. 16B) for 72 h, and T cell proliferation in each culture was measured using [³H]-thymidine incorporation assay. The cell proliferation is shown as stimulation index (SI; mean±SD) of four independent experiments (*p<0.05, CD26^{+/+} vs. CD26^{-/-} mice). [³H]-thymidine incorporation in lymph node cells stimulated with 50 μg/ml MOG p35-55 was determined as 10,332±4,590 cpm (CD26^{+/+}) and 45,180±7,530 cpm (CD26^{-/-}) and in PWM-stimulated lymph node cells as 80,482±30, 130 cpm (CD26^{+/+}) and 122,133±37, 608 cpm (CD26^{-/-}).

Cytokine measurements. For determination of cytokine secretion, lymph node cells or splenocytes are cultured in AIM-V medium supplemented with 10⁻⁵ M 2-ME. Cells are stimulated with anti-CD3 and anti-CD28. Cell culture supernatants are harvested after 48 h and stored at −70° C. until cytokine determination. TNF-α, IFN-γ, IL-2, IL-4 and IL-10 concentrations of cell culture supernatants are determined with specific enzyme-linked immunosorbent assays according to standard procedures (FIGS. 16C-H). In FIGS. 16C-16H, T cells were isolated from regional draining lymph nodes of wild-type (CD26^{+/+}) and CD26^{-/-} mice on day 11 or 12 following immunization with MOG p35-55. MOG-primed lymphocytes were cultured in presence of different concentration of MOG p35-55. Cell culture supernatants were collected 48 h later. Levels of IFN-g (FIG. 16C), IL-2 (FIG. 16D), TNF-a (FIG. 16E), IL-4 (FIG. 16F), IL-10 (FIG. 16G), and latent TGF-b1 (FIG. 16H) in the cultures were measured by specific ELISA. Cytokine production is shown as mean±SEM of four independent experiments (*p<0.05). PDE inhibitors are added in selected cultures in concentrations as indicated in Table 3.

Intracellular cytokine staining. As an alternative approach, intracellular cytokine staining for IFN-γ and IL-17 is determined, a recently discovered ‘signature’ cytokine for autoimmune T cells (Langrish, et al., J Exp Med, vol. 201:233-240 (2005); Cua, et al., Nature, vol. 421:744-748 (2003); and Hofstetter et al., J Neuroimmunol, vol. 170:105-114 (2005)). Cells are washed twice in PBS, fixed with paraformaldehyde (2%), and then permeabilized using saponin (0.5%) for intracellular staining. The following mAbs are used: PE-coupled rat anti-mouse IL-17 (TC11-18H10) titrated to >0.25 μg mAb/10⁶ cells; FITC-coupled anti-IFN-γ (XMG1.2) titrated to >0.5 μg mAb/10⁶ cells. The level of background staining is assessed using rat IgG1 (PE-R3-34) and rat IgG1 (FITC-R3-34) at >0.25 μg/10⁶ cells and >0.5 μg/10⁶ cells, respectively.

Isolation of T cell subpopulations. CD4⁺ and CD8⁺ T cell populations are isolated from lymph node cell cultures using commercial kits based on magnetic bead assays according to standard procedures.

Flow chamber assays. To test PDE control of adhesion strength of activated T cells to VCAM-1, a flow chamber assay system is used. (See e.g., Cinamon and Alon, Methods Mol Biol, vol. 239:233-242 (2004); Cinamon and Alon, J Immunol Methods, vol. 273:53-62 (2003); and Cinamon et al., Nat Immunol, vol. 2:515-522 (2001)) Recombinant mouse VCAM-1 is mixed in coating medium (PBS buffered with 20 mM sodium bicarbonate pH, 8.5) with a fixed amount of carrier (2 mg/ml HSA) and adsorbed on polystyrene plates for 2 h at 37° C., alone or with the indicated amounts of intact or heat-inactivated chemokines. Plates are washed and blocked with HSA (20 mg/ml). VCAM-1/chemokine-coated substrates are assembled as the lower wall of the flow chamber (260-mm gap) and extensively washed with binding medium. The flow chamber is mounted on the stage of an inverted phase contrast microscope. All flow experiments are conducted at 37° C. T cells are perfused at 10⁶ cells/ml through the chamber at indicated flow rates generated with an automated syringe pump. The entire period of cell perfusion is recorded on videotape with a long integration CCD video camera and a time-lapse recording program. All cellular interactions with the adhesive substrates are determined by manually tracking the motions of individual cells along 0.9-mm field paths for 1 min.

To test PDE control of vascular recruitment of activated T cells, a model barrier of bEND.3 cells grown to confluence on fibronectin coated polystyrene dishes is used. To induce the critical adhesion molecule VCAM-1, the monolayers are activated with TNF-α (2 μg/ml) for 2 h. Following activation, the dish is secured on the lower level of a parallel plate laminar flow apparatus which creates a 260 μM vertical gap). Once assembled, CXCL12 (100 ng/ml) or CCL19 (100 ng/ml) is perfused over the monolayer for 5 min to immobilize chemokine followed by perfusion of buffer to remove unabsorbed chemokine from the chamber system. Purified T cell population of a previously determined type is then allowed to accumulate under a low shear stress of 0.75-1.5 dyn/cm² for 40-120 s followed by an increased shear stress to 2-10 dyn/cm² for 10-20 min. Shear stress is generated by an automated syringe pump attached to the chamber outlet port. The perfusion period is recorded by real-time videomicroscopy, during accumulation, and time-lapse videomicroscopy, during the higher shear stress period. Motion analysis is then performed manually on the time-lapse video according to the following criteria: (I) initial tethers (defined as T cells that make rolling or stationary contact with the endothelial surface during the period of observation), (II) firm stationary adhesion (defined as T cells that make initial tethers and then adhere firmly, remaining fixed for the duration of the assay), (III) locomotion of cells on the apical endothelial monolayer (defined as T cells that form non-stationary firm adhesion to the endothelium), and (IV) transmigration through the monolayer (defined as T cells that made initial tethers, firmly adhere to the endothelium, undergo locomotion, and then migrate beneath the surface of the endothelium through an intercellular junction).

Immunoblot. Analysis of PDE expression by Western Immunoblot methodology is performed as described in a recent publication (Tiwari, et al., Biochem. Pharmacol, vol. 69:473-483 (2005)). Primary antibodies against each of the 21 known genes encoding PDEs are available commercially, e.g., from FabGennix Inc. (Shreveport, La.).

Peptides. Myelin oligodendrocyte glycoprotein peptide 35-55 (MOG p35-55), corresponding to mouse sequence (MEVGWYRSPFSRVVHLYRNGK, SEQ ID NO:9), is synthesized on a peptide synthesizer by standard 9-fluorenylmethoxycarbonyl chemistry, and purified by high-performance liquid chromatography (HPLC).

Induction of active EAE and clinical evaluation. Active EAE is induced in 8-12 weeks old mice by immunization with MOG p35-55 in complete Freund's adjuvant. 200 μg of MOG p35-55 peptide and 800 μg of killed mycobacterium tuberculosis are emulsified in CFA and injected subcutaneously by means of four injections over the flanks. In addition, 200 ng of pertussis toxin dissolved in 200 μl PBS is injected i.p. at the day of immunization and again the day after. Mice are monitored daily for clinical signs of EAE, and scored according to the following criteria: 0, no signs of disease; 0.5, partial tail weakness; 1, limp tail; 1.5, limp tail and slight slowing of righting; 2, partial hind limb weakness and/or marked slowing of righting; 2.5, dragging of hind limb(s) without complete paralysis; 3, complete paralysis of at least one hind limb; 4, severe forelimb weakness; 5, moribund or dead. Daily clinical scores are calculated and presented as the average (mean) and standard error of the mean (SEM) of all individual disease scores in each group. Statistical comparison of disease severity by clinical score is accomplished by calculating the mean clinical score for each mouse from day of disease onset to day 60 and performing a non-parametric Wilcoxon analysis.

CD26^{-/-} mice show significantly higher clinical and histopathologic severity scores of EAE compared to wt mice. (A) Disease course of EAE (mean score±SEM) in wt (CD26^{+/+}) and CD26^{-/-} mice is plotted from day 0 to day 38 (each group n=27 combined from 3 independent experiments; p=0.048, CD26^{+/+} vs. CD26^{-/-} mice). The insert shows incidence of EAE in the experimental groups. (B) Histopathologic analysis revealed an increased number of meningeal (M), parenchymal (P) and total (T) inflammatory foci in CD26^{-/-} mice compared to wild-type (CD26^{+/+}) mice. Data represent mean number of inflammatory foci±SEM. The differences in the number of foci between the two groups (each n=4) was statistically significant with p=0.0003 (M), p=0.0097 (P) and p=0.0023 (T).

Histopathology. These studies focus on scoring of inflammatory infiltrates in EAE. Selected mice are perfused with 20 ml cold PBS on day 14 after immunization. Brains and spinal cords are extracted, fixed in 4% (w/v) paraformaldehyde and embedded in paraffin. Sections were stained with haematoxylin and eosin. Brain, thoracic and lumbar spinal cord sections are evaluated and meningeal, parenchymal and total numbers of inflammatory foci are determined by an examiner blinded to the treatment status of the animal.

Statistical analysis of EAE severity: Statistical comparison of clinical EAE disease severity is accomplished by performing a Wilcoxon analysis for non-parametric data sets using SigmaStat software. Histopathological data are analyzed with using the unpaired two-tailed Student's t-test using SigmaStat software.

### Example 2

### CXCL12 Induces Migration of Murine Splenocytes

The studies provided herein demonstrated that cAMP modulation of T cell migration indicates different intracellular regulation between stimulated and unstimulated cells. Directed migration of T cells to specific tissues is believed to play an important role in lesion formation associated with inflammatory diseases. To investigate cAMP signaling and PDE control of T cell migration, broad modulators of the synthetic and degradative enzymes that regulate cAMP were used in chemotaxis assays. Previous studies had shown that chemotaxis of human T cells to several stimuli, including CXCL12, could be inhibited by agents known to stimulate the cAMP signaling pathway. T cells used in these previous chemotaxis studies were, however, quiescent, unstimulated cells. Inasmuch as it is now well accepted that pro-inflammatory T cell populations that participate in transendothelial migration and enter sites of inflammation represent activated effector/memory T cells with a Th1/Th17 phenotype (Langrish, et al., J Exp Med, vol. 201:233-240 (2005); Cua, et al., Nature, vol. 421:744-748 (2003); and Hofstetter et al., J Neuroimmunol, vol. 170:105-114 (2005)), unstimulated and ConA-stimulated splenocytes were compared for modulation of their chemotactic response by agents that stimulate the cAMP signaling pathway.

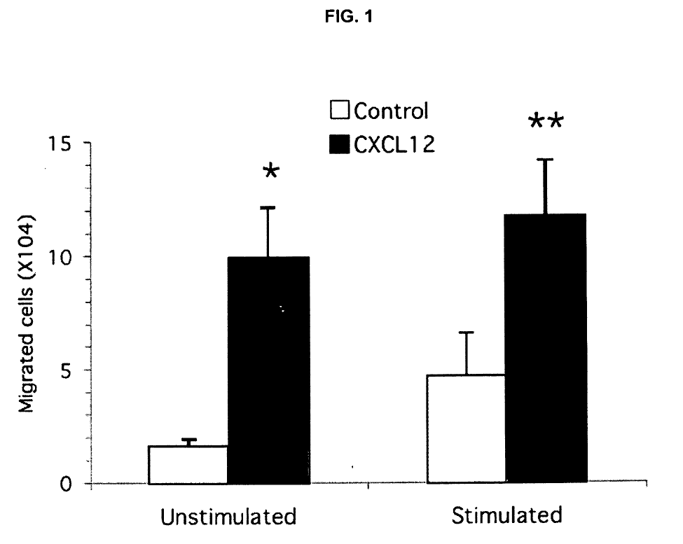

Using the transwell assay system, CXCL12 (250 ng/ml) stimulated the migration of both unstimulated and Con A-stimulated mouse splenocytes (FIG. 1). Mouse splenocytes (3×10⁵), isolated as indicated in the Methods described above, were placed in the upper chamber insert of 24 well transwell plates and the number of cells migrating to the lower chamber were counted after 4 hr, as described in the Methods. Where included, CXCL12 (250 ng/ml) was added to the lower chamber only. Results represent the mean±S.D. of four separate experiments with experimental points assayed in triplicate. *p<0.001, **p<0.02.

In response to CXCL12, there was a 6.2-fold increase in the number of unstimulated splenocytes and a 2.5-fold increase in the number of Con A-stimulated splenocytes migrating to the side of the chamber containing CXCL12. The number of cells migrating to the chamber containing CXCL12 was about the same for unstimulated and Con A-stimulated splenocytes. The difference in fold stimulation between the two cell populations was primarily due to increased migration of Con A-stimulated splenocytes, relative to unstimulated splenocytes, in the absence of CXCL12.

### Example 3

### Effect of cAMP Analogue Adenylyl Cyclase Activator and PDE Inhibitors on CXCL12 Induced Splenocyte Chemotaxis

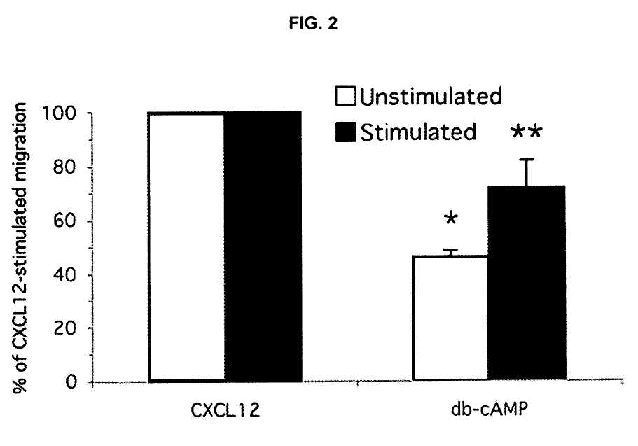

The cell permeable cAMP analogue, dibutyryl cAMP (500 μM) significantly inhibited CXCL12-induced migration of both unstimulated and Con A-stimulated splenocytes by 54% and 29% respectively (FIG. 2). Splenocytes isolated from mice were assayed for migration in response to CXCL12 (250 ng/ml), either directly (unstimulated) or following 48 hr of incubation with 3 μg/ml Con A (stimulated), as described in Methods. To test for effects of dibutyryl cAMP, cells were pretreated with dibutyryl cAMP (500 μM) for 45-60 min prior to beginning the chemotaxis assay, and the assays were conducted with dibutyryl cAMP (db-cAMP) present (500 μM) in both the upper and lower chambers of the transwell plates. Data plotted are derived from a single experiment performed in triplicate. *p<0.001; **p<0.02.

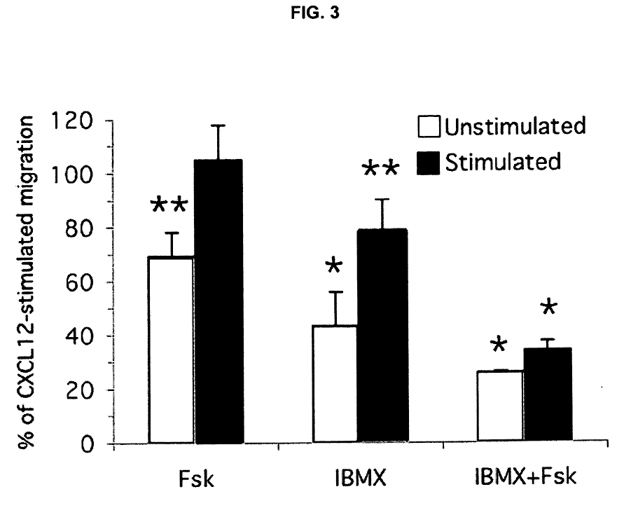

In contrast, the responses of these two cell populations, the unstimulated and Con A-stimulated splenocytes, to adenylyl cyclase activation or PDE inhibition were quite different. Forskolin (25 μM), a direct activator of adenylyl cyclase, inhibited CXCL12-induced splenocyte migration by 31%, but had no effect at all on CXCL12-induced migration of Con A-stimulated splenocytes (FIG. 3). Chemotaxis assay conditions were the same as those described above in connection with FIG. 2 except that the test agents used were forskolin (Fsk) (25 μM) and IBMX (300 μM). Results represent the mean±S.D. of four separate experiments assayed in triplicate. *p<0.005; **p<0.05.

Similar to what was seen with activation of adenylyl cyclase, the non-specific PDE inhibitor, IBMX (300 μM) also produced a differential effect on CXCL12-induced migration of unstimulated and Con A stimulated splenocytes. Whereas IBMX inhibited migration of unstimulated splenocytes by 57%, it only inhibited Con A-stimulated splenocyte migration by 21% (FIG. 3). When IBMX and forskolin were added together, however, greater inhibition was seen. In the presence of both forskolin and IBMX, migration of unstimulated splenocytes was inhibited by 74% and Con A-stimulated splenocytes by 66%.

Response of Con A-stimulated splenocytes to PDE gene family selective inhibitors was also examined. Chemotaxis assay conditions were the same as those described above in connection with FIG. 2 except that only Con A-stimulated splenocytes were used and the test agents used were the PDE3-selective inhibitor motapizone (10 μM), the PDE4-selective inhibitor piclamilast (1 μM) and a PDE7-selective inhibitor (not shown). Data plotted are derived from a single experiment performed in triplicate. There was no statistically significant effect of any of these PDE inhibitors in this assay.

Thus, whereas the cell permeable cAMP analogue, dibutyryl cAMP, readily inhibited chemotaxis of both cell populations, the adenylyl cyclase activator, forskolin, and the general non-selective PDE inhibitor, IBMX, inhibited migration of unstimulated splenocytes, but either had no effect at all (forskolin) or only a limited effect (IBMX) on activated cells, except when added together. Since direct addition of an analogue of cAMP inhibits migration of both cell types, whereas broad modulators of the synthetic and degradative enzymes that regulate cAMP primarily inhibited migration only of unstimulated cells, it was concluded that unstimulated and activated splenocytes must differ in the way in which they regulate cAMP within the cell.

### Example 5

### Effect of PDE Inhibitors on Adhesion and Migration of Activated T Cells

In the studies described herein, dipyridamole, a PDE inhibitor capable of inhibiting PDE8, blocked adhesion and migration of activated T cells, but selective PDE4 inhibitors did not.

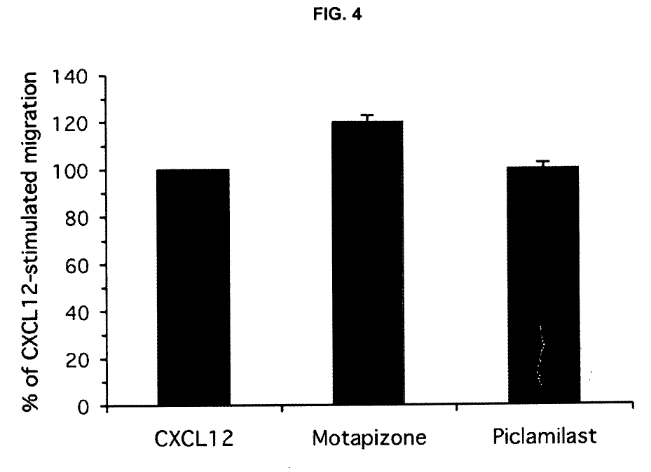

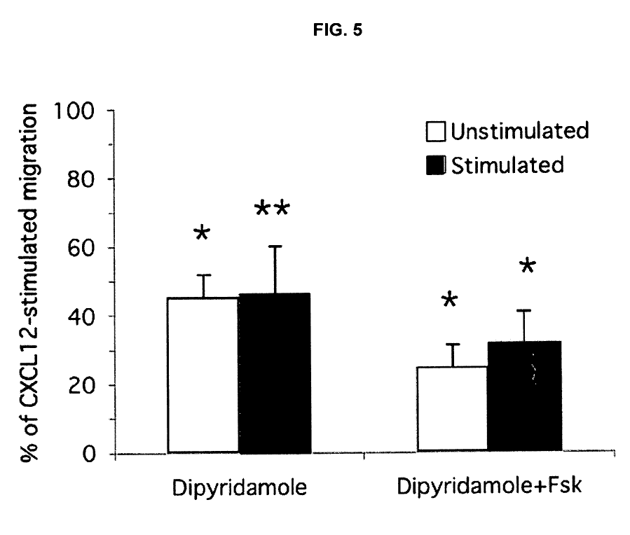

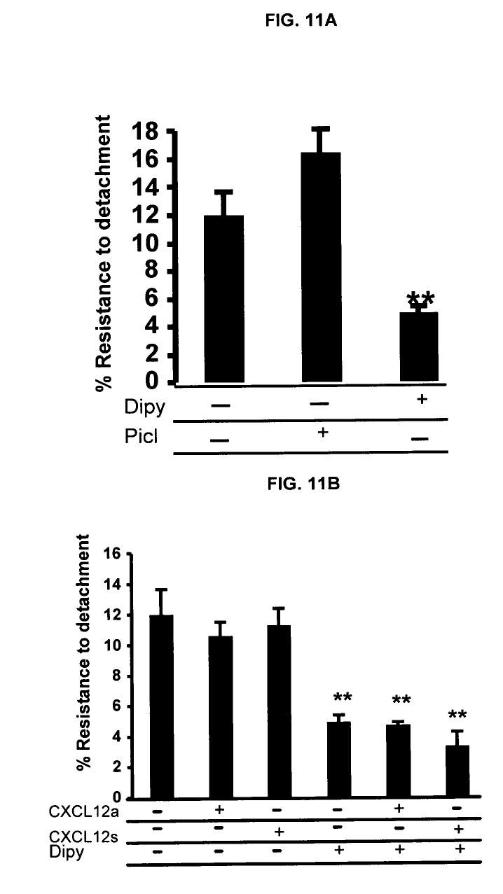

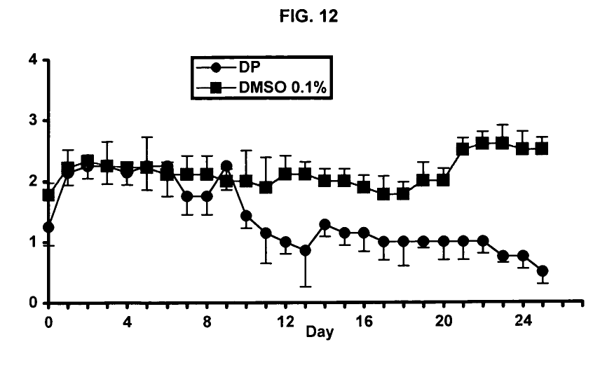

In inflammation, T cell homing to most target tissues requires an initial tethering step that leads to rolling in postcapillary venules followed by a chemokine dependent activation step which triggers firm adhesion and T cell emigration (diapedesis or transendothelial migration (TEM)). To determine PDE control of T cell adhesion to brain derived endothelium and chemotaxis, PDE inhibitors were tested in vitro using adhesion (FIG. 11) and chemotaxis assays (FIGS. 4 and 5). It has become increasingly apparent in recent years that in response to stimuli, cAMP elevations in cells occur in a directed spatial and temporal manner, resulting in the formation of microdomains of localized cAMP concentrations within the cell, and it is the regulation of these localized domains of cAMP in physiologically important compartments that regulate specific functions of the cell (Rich, et al., Proc Natl Acad Sci USA, vol. 98:13049-13054 (2001)). It is now established that one way microdomains of localized cAMP concentrations are achieved in the cell is through selective expression and compartmentalization of different isoforms of PDEs (Mongillo, et al. Circ Res, vol. 95:67-75 (2004); Rich, et al., J Gen Physiol, vol. 118:63-78 (2001) and Brunton, L. L. 2003. PDE4: arrested at the border. Sci STKE 2003:PE44.). Since migration of unstimulated splenocytes was readily inhibited by the general non-selective PDE inhibitor, IBMX, and activated splenocytes were far less affected by IBMX, it was hypothesized that activated splenocytes express an IBMX insensitive PDE activity in a functionally relevant cell compartment linked to regulation of cell migration and adhesion, which limits the accumulation of cAMP in that compartment in response to activators of adenylyl cyclase, IBMX or PDE selective inhibitors targeted to IBMX-sensitive PDE gene families. The non-specific PDE inhibitor IBMX inhibits all known PDE gene families capable of hydrolyzing cAMP with the possible exception of PDE8, since expressed forms of full length cDNAs for PDE8 have been reported to be resistant to IBMX inhibition (Soderling et al., Proc Natl Acad Sci USA, vol. 95:8991-8996 (1998); Fisher et al., Biochem Biophys Res Commun, vol. 246:570-577 (1998); and Gamanuma, et al., Cell Signal, vol. 15:565-574 (2003)). Among a wide variety of PDE inhibitors tested against expressed forms of PDE8A, only dipyridamole was found to inhibit this enzyme, with reported IC₅₀s in the range of 4-9 μM (Soderling et al., Proc Natl Acad Sci USA, vol. 95:8991-8996 (1998); Fisher et al., Biochem Biophys Res Commun, vol. 246:570-577 (1998); and Gamanuma, et al., Cell Signal, vol. 15:565-574 (2003)). Therefore, in order to determine the effects of inhibition of PDE8 on splenocyte adhesion and migration, the effects of dipyridamole were tested in these systems.

The hypothesis was first tested using a series of experiments determining T cell adhesion to vascular endothelial cells. Confluent bEND.3 brain derived endothelial cell monolayers were pretreated with 100 ng/ml TNF-α. Con A activated splenocytes (5×10⁶ cells/ml) were labeled with 5 μM Calcein AM for 30 min at 37° C. and 7×10⁵ splenocytes were suspended in medium in the presence of dipyridamole or vehicle control and added to each well. Splenocytes were allowed to adhere for 30 min at 37° C. without shaking after which nonadherent cells were removed by washing the wells 4× with prewarmed D-PBS. For analysis, 7×10⁵ calcein AM labeled cells were added to an empty well as a representation of maximal possible fluorescence. The fluorescence of each well was read by a Victor 3v microplate reader with a fluorescein filter set. Background fluorescence (medium only) was subtracted from each well and percentage of adherent cells that is resistant to detachment under the conditions of washing the wells was calculated as fluorescence of well in percent of maximal fluorescence (without washing).

As shown in FIGS. 11A and 11B, adhesion to endothelial cells of Con A-stimulated splenocytes was not inhibited at all by the PDE4-selective inhibitor, piclamilast (1 μM). This concentration of the PDE4 family specific PDE inhibitor piclamilast used maximally inhibited the PDE family it targets without losing selectivity for inhibition of that given PDE gene family. Additionally, a PDE7-selective inhibitor had no significant effect on adhesion of Con A-stimulated splenocytes as well (data not shown). Among a wide variety of PDE inhibitors tested against expressed forms of PDE8A, only dipyridamole was found to inhibit this enzyme, with reported IC₅₀s in the range of 4-9 μM (Soderling et al., Proc Natl Acad Sci USA, vol. 95:8991-8996 (1998); Fisher et al., Biochem Biophys Res Commun, vol. 246:570-577 (1998); and Gamanuma, et al., Cell Signal, vol. 15:565-574 (2003)). Therefore, in order to determine the effects of inhibition of PDE8 on splenocyte adhesion, the effects of dipyridamole were tested in this system. As shown in FIGS. 11A and 11B, dipyridamole (100 μM) inhibited adhesion of Con A-stimulated splenocytes by 50-54%. The percent of cells resistant to detachment was determined by fluorometer (**p<0.001). Then, tests were conducted to determine whether the reduction in adhesion of activated splenocytes by dipyridamole could be reversed by an additional chemokine signal. Besides its chemotactic properties, CXCL12 is known to promote T cell adhesion to vascular endothelium and TEM under flow conditions. However, when used in the static assay system described herein, addition of CXCL12 did not increase T cell resistance to detachment, nor did it overcome the inhibition by dipyridamole (FIG. 11B). The presentation of CXCL12 had no influence on these results as both CXCL12 absorbed (CXCL12a) to endothelium as well as soluble CXCL12 (CXCL12s) did not enhance splenocyte attachment to the bEND.3 cell monolayer.

Next, in order to determine the effects of inhibition of PDE8 on splenocyte chemotaxis, the effects of dipyridamole were tested in this system. As shown in FIG. 4, migration of Con A-stimulated splenocytes was not inhibited at all by the PDE3-selective inhibitor, motapizone (10 μM), nor by the PDE4-selective inhibitor, piclamilast (1 μM). These concentrations of the family specific PDE inhibitors used also maximally inhibit the PDE family they target without losing selectivity for inhibition of that given PDE gene family (Lerner and Epstein. Biochem J, vol. 393:21-41 (2006) and Tenor, et al., Clin Exp Allergy, vol. 25:616-624 (1995)). Additionally, a PDE7-selective inhibitor had no significant effect on migration of Con A-stimulated splenocytes as well (data not shown). Of note, in addition to the relative insensitivity to IBMX, migration of activated splenocytes was also found to be resistant to inhibition by gene family specific inhibitors targeted to PDE3, PDE4, and PDE7. The inhibition of stimulated splenocytes seen following combined addition of forskolin and IBMX could result from excessive increases in cAMP causing spillover from one cellular compartment to another, and overwhelming the normal cAMP degradative system in the compartment responsible for regulation of migration. The non-specific PDE inhibitor IBMX inhibits all known PDE gene families capable of hydrolyzing cAMP with the possible exception of PDE8, since expressed forms of full length cDNAs for PDE8 have been reported to be resistant to IBMX inhibition (Soderling et al., Proc Natl Acad Sci USA, vol. 95:8991-8996 (1998); Fisher et al., Biochem Biophys Res Commun, vol. 246:570-577 (1998); and Gamanuma, et al., Cell Signal, vol. 15:565-574 (2003)).

As only dipyridamole was found to inhibit PDE8A (Soderling et al., Proc Natl Acad Sci USA, vol. 95:8991-8996 (1998); Fisher et al., Biochem Biophys Res Commun, vol. 246:570-577 (1998); and Gamanuma, et al., Cell Signal, vol. 15:565-574 (2003)), the effect of dipyridamole on splenocyte migration was tested. As shown in FIG. 5, dipyridamole (100 μM) inhibited the CXCL12-induced migration of both unstimulated and Con A-stimulated splenocytes by 55% and 54%, respectively. Addition of forskolin (25 μM) along with dipyridamole further potentiated the inhibition of migration of the two cell populations to 76% and 68%, respectively (FIG. 5). Dipyridamole inhibition of CXCL12-stimulated chemotaxis was reversed by the cAMP-dependent protein kinase (PKA) inhibitor, Rp-cAMPS. Dipyridamole and rolipram were shown to inhibit lipopolysaccharide-stimulated release of TNF-α from peripheral blood mononuclear cells (Eigler et al., Scand J Immunol 45:132-139 (1997)). Addition of adenosine deaminase to the cultures potentiated the release of TNF-α and attenuated the inhibition of TNF-α release by rolipram, suggesting that the effects of these agents on TNF-α release in this system are mediated, at least in part, through extracellular adenosine that accumulates in the cultures (Eigler et al., Scand J Immunol 45:132-139 (1997)).

The effects of adenosine deaminase on CXCL12-stimulated splenocyte migration and its inhibition by dipyridamole were tested. Adenosine deaminase by itself had no effect on splenocyte migration (not shown), nor did it affect the dipyridamole inhibition of migration. Thus, in support of the hypothesis, it was found that dipyridamole profoundly inhibited chemotactic migration of both unstimulated and stimulated splenocytes and that its actions appeared to be independent of its ability to inhibit adenosine uptake. Moreover, when forskolin was added along with dipyridamole, to stimulate adenylyl cyclase, inhibition of migration was potentiated, leading to as much as 70-80% inhibition of migration of both unstimulated and stimulated cells, further suggesting that the effects of dipyridamole on chemotaxis are mediated by its stimulation of the cAMP signaling pathway through inhibition of PDE. The concept that dipyridamole is working through a cAMP mediated effect is also borne out by the reversal of the dipyridamole effect that is seen with the PKA antagonist, Rp-cAMPS.

The non-specific PDE inhibitor IBMX inhibits all known PDE gene families capable of hydrolyzing cAMP with the possible exception of PDE8, since expressed forms of full length cDNAs for PDE8 have been reported to be resistant to IBMX inhibition (Soderling, et al., Proc Natl Acad Sci USA, vol. 95: 8991-8996 (1998); Fisher, et al., Biochem Biophys Res Commun, vol. 246: 570-577 (1998); and Gamanuma, et al., Cell Signal, vol. 15: 565-574 (2003)). Among a wide variety of PDE inhibitors tested against expressed forms of PDE8A, only dipyridamole was found to inhibit this enzyme, with reported IC50s in the range of 4-9 μM (Soderling, et al., Proc Natl Acad Sci USA, vol. 95: 8991-8996 (1998); Fisher, et al., Biochem Biophys Res Commun, vol. 246: 570-577 (1998); and Gamanuma, et al., Cell Signal, vol. 15: 565-574 (2003)). Therefore, in order to determine the effects of inhibition of PDE8 on splenocyte migration, the effects of dipyridamole were tested in this system. Chemotaxis assay conditions were the same as those described above in connection with FIG. 2 except that the test agents used were dipyridamole (100 μM) and dipyridamole (100 μM)+forskolin (Fsk) (25 μM). Results represent the mean±S.D. of four separate experiments assayed in triplicate. *p<0.002; **p<0.05.

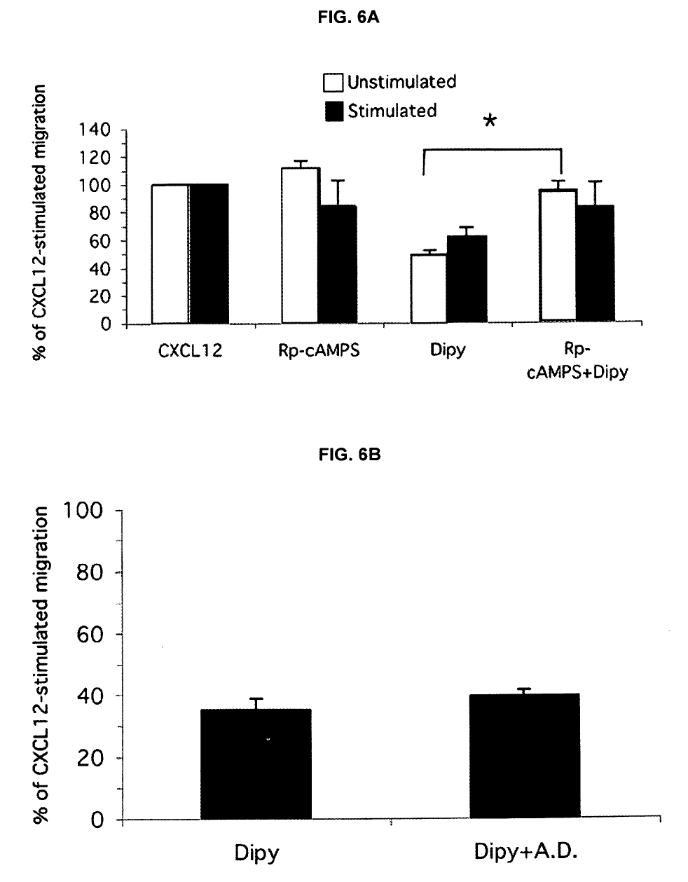

As shown in FIG. 5, dipyridamole (100 μM) inhibited the CXCL12 induced migration of both unstimulated and Con A-stimulated splenocytes by 55% and 54% respectively. Addition of forskolin (25 μM) along with dipyridamole further potentiated the inhibition of migration of the two cell populations to 76% and 68% respectively (FIG. 5). Dipyridamole inhibition of CXCL12-stimulated chemotaxis was reversed by the cAMP-dependent protein kinase (PKA) inhibitor, Rp-cAMPS (FIG. 6A). To determine the effect of Rp-cAMPS, chemotaxis assay conditions were the same as those described above in connection with FIG. 2 except that the test agents used were RpcAMPS (1 mM) and dipyridamole (Dipy) (100 μM). Results represent the mean±S.D. of two separate experiments assayed in triplicate. *p<0.001.

Dipyridamole and rolipram were shown to inhibit lipopolysaccharide-stimulated release of tumor necrosis factor (TNF) from peripheral blood mononuclear cells (Eigler, et al., Scand J Immunol, vol. 45: 132-139 (1997)). Addition of adenosine deaminase to the cultures potentiated the release of TNF and attenuated the inhibition of TNF release by rolipram, suggesting that the effects of these agents on TNF release in this system are mediated, at least in part, through extracellular adenosine that accumulates in the cultures (Eigler, et al., Scand J Immunol, vol. 45: 132-139 (1997)). The effects of adenosine deaminase on CXCL12-stimulated splenocyte migration and its inhibition by dipyridamole were tested. Adenosine deaminase by itself had no effect on splenocyte migration (not shown), nor did it affect the dipyridamole inhibition of migration (FIG. 6B). In the determination of the effect of adenosine deaminase, results shown represent stimulated splenocytes. Test reagents used were dipyridamole (Dipy) (100 μM) and adenosine deaminase (A.D.) (1 U/ml).

### Example 5

### Expression of Splenocyte mRNA for PDE4B2, PDE7A1, and PDE8A1 Following Con A Stimulation

Previous studies focused on PDE4 isoforms as intracellular targets for therapies in chronic inflammatory diseases. However, dose-limiting side effects in humans, of which nausea and vomiting are the most common, have hampered the clinical success of PDE4 isoforms. Thus, the studies presented herein were carried out to overcome these limitations by identifying other PDE isoforms that are expressed in immune cells and inhibition of these isoforms provides the same or better therapeutic activity as the PDE4 isoforms alone, but does not incur dose-limiting side effects. In the initial step of a complete analysis of PDE expression patterns and functions in inflammatory cells, the expression of splenocyte mRNA for PDE4B2, PDE7A 1, and PDE8A1 following Con A stimulation of mouse splenocytes was investigated.

The insensitivity of Con A-stimulated splenocytes to adenylyl cyclase activators and PDE inhibitors other than dipyridamole, relative to unstimulated cells, could be explained by an upregulation of PDE8 following Con A stimulation, which would prevent cAMP accumulation in activated cells. In order to examine this possibility, quantitative real-time RT-PCR was performed to look at mRNA levels of PDE4B2, PDE7A1, and PDE8A1 following Con A stimulation. Quantitative real-time RT-PCR was performed on mouse splenocytes at different times following Con A stimulation as described in the Methods provided above. Relative expression of mRNA was calculated as follows: (amplification number of target gene/amplification number of RPL19 housekeeping gene at a given time point)/(that for zero time). Results represent the mean±S.D. of three separate experiments assayed in triplicate. *p<0.05, **p<0.01.

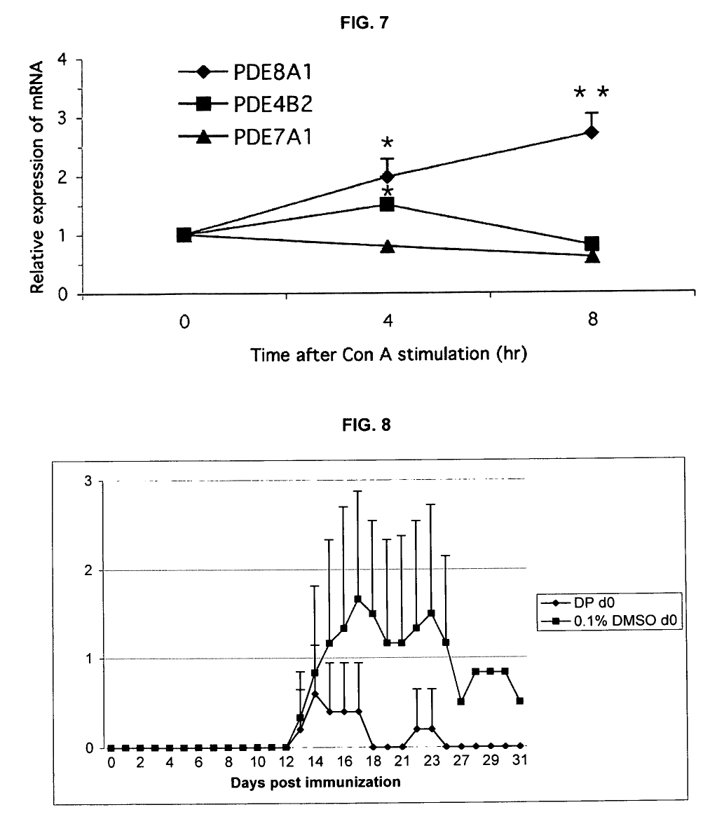

As shown in FIG. 7, mRNA for PDE8A1 is induced 2.7-fold within 8 hr following Con A stimulation. In contrast, PDE7A1 mRNA did not increase at all following Con A stimulation, and PDE4B2 mRNA increased by only 1.5-fold at 4 hr and then returned to baseline by 8 hr. Thus, as in human T cells, PDE8A1 is induced upon activation in mouse T cells and is a target to suppress the function of activated cells.

### Example 6

### The Effect of Dipyridamole (DP) Treatment on EAE

The studies provided herein demonstrated that dipyridamole suppressee clinical signs of inflammation in vivo. The therapeutic potential of selective PDE4 inhibitors in Th1/Th17-mediated inflammatory diseases (Langrish, et al., J Exp Med, vol. 201:233-240 (2005); and Cua, et al., Nature, vol. 421:744-748 (2003)) has been widely studied (Giembycz, Proc Am Thorac Soc, vol. 2:326-333; discussion 340-321 (2005); Giembycz, Curr Opin Pharmacol, vol. 5:238-244 (2005); Bender and Beavo. Pharmacol Rev, vol. 58:488-520 (2006); Bernareggi, et al., Br J Pharmacol, vol. 128:327-336 (1999); Giembycz, Br J Clin Pharmacol, vol. 62:138-152 (2006); Sommer et al., J Neuroimmunol, vol. 79:54-61. (1997); Jung et al., J Neuroimmunol, vol. 68:1-11 (1996); Moore et al., J Pharmacol Exp Ther, vol. 319:63-72 (2006); Martinez et al., Brain Res, vol. 846:265-267 (1999); Bielekova, et al., J Immunol, vol. 164:1117-1124 (2000); Lagente et al., Mem Inst Oswaldo Cruz, vol. 100 Suppl 1:131-136 (2005); and Ouagued et al., Pulm Pharmacol Ther, vol. 18:49-54 (2005)). While successful preventive treatment is well documented, data on reversal of chronic inflammation are less conclusive (Giembycz, Curr Opin Pharmacol, vol. 5:238-244 (2005); Smith and D. Spina, Curr Opin Investig Drugs, vol. 6:1136-1141 (2005); Dyke and Montana, Expert Opin Investig Drugs, vol. 11:1-13 (2002); Huang, et al., Curr Opin Chem Biol, vol. 5:432-438 (2001); Essayan, Biochem Pharmacol, vol. 57:965-973 (1999); Kroegel and Foerster, Expert Opin Investig Drugs, vol. 16:109-124 (2007); Folcik et al., J Neuroimmunol, vol. 97:119-128 (1999); and Dinter et al., J Neuroimmunol, vol. 108:136-146 (2000)). To study the role of PDE8 in inflammation in vivo, experimental autoimmune encephalomyelitis (EAE) was induced in C57BL/6 mice, an animal model widely used for the study of the pathogenesis and therapy of the human disease multiple sclerosis (Brocke, et al., Nature, vol. 379:343 (1996); Brocke et al., Proc Natl Acad Sci USA 96:6896 (1999); and Steinbrecher, et al., J Immunol 166:2041 (2001)). EAE models can be performed using mutant and conventional mice (Liblau et al., Int Immunol, vol. 9:799-803 (1997); Preller et al., J Immunol, vol. 178(7):4632-40 (2007); and Ferber et al., J Immunol, vol. 156:5-7 (1996)).

From day 0 until day 23, mice were injected with 2 mg of the PDE inhibitor DP per mouse per day or 0.1% DMSO in PBS as vehicle control as indicated in FIG. 8. DP is the only inhibitor known to block PDE 8 in addition to other PDEs. When EAE developed following immunization with myelin oligodendrocyte glycoprotein peptide 35-55, mice showed paralytic signs of the disease and were scored (EAE score) according to standard protocols (Brocke, et al., Nature, vol. 379:343 (1996); and Brocke et al., Proc Natl Acad Sci USA 96:6896 (1999)). Average EAE scores+SD values of all mice in each experimental group are shown.

Daily injections of DP profoundly suppressed the development of clinical signs of EAE in immunized mice. These data suggest that targeting PDE8 is a promising approach for the development of treatments in inflammatory autoimmune or allergic diseases such as multiple sclerosis, rheumatoid arthritis, asthma and inflammatory bowel disease.

To assess whether dipyridamole prevented EAE progression after the first signs of clinical disease onset, dipyridamole (1 mg/mouse/day i.p. for the first 8 days and 2×1 mg/mouse/day i.p. from day 9 to day 22) or vehicle control (0.1% DMSO in PBS) were administered once the mean clinical EAE scores reached grade 1. Dipyridamole given at a dose of 2×1 mg/mouse/day i.p., but not a single dose of 1 mg/mouse/day i.p., significantly decreased the clinical severity of EAE compared with vehicle control treatment.

### Example 7

### Identification of PDE Targets

The studies described herein are used to identify novel PDE targets in anti-CD3 and anti-CD28 stimulated T cells treated with selective PDE inhibitors from wildtype and PDE mutant subjects such as mice.

The studies described herein are carried out to test the hypothesis that if activated T cells contribute to the pathogenesis of smoking associated inflammation in vivo, then suppression of these T cells should improve or cure these inflammatory conditions. It is clear from a number of clinical trials with PDE4 inhibitors that targeting of activated T cells in vivo is not a trivial task, especially once they have been activated and have become effector/memory T cells. PDE4 activity accounts for the majority of the total PDE activity in T cells, and selective PDE4 inhibitors showed great therapeutic efficacy in animal studies. Despite these observations, when used in clinical trials for asthma and other inflammatory illnesses, PDE4 inhibitors showed limited success, and consequently, none have yet been approved for clinical use. A possible explanation for the limited efficacy of selective PDE4 inhibitors in clinical trials is provided by early findings of PDE enzymatic activity in resting T cells in which isoforms in addition to PDE4 were shown to be expressed (FIG. 10.; Table 1) (Lerner and Epstein. Biochem J, vol. 393:21-41 (2006) and Tenor, et al., Clin Exp Allergy, vol. 25:616-624 (1995)).

TABLE 1
PDE isoforms as potential regulators of cAMP signaling in immune cells

| PDE Isoform | Specificity | cAMP Km | | cGMP Km | Immune cell expression |
|---|---|---|---|---|---|
| 1B1 | cAMP and cGMP | 7-24 | μM | 3 μM | Activated T cells |
| | | | | | Dendritic cells |
| 1B2 | cAMP and cGMP | 7-24 | μM | 3 μM | Activated T cells |
| | | | | | Macrophages |
| 3B | cAMP and cGMP | 0.2-0.5 | μM | 0.02-0.2 μM | Peripheral blood T cells |
| | | | | | Peripheral blood B cells |
| | | | | | Macrophages |
| 4A | cAMP | 1-4 | μM | N/A | Activated T cells |
| 4B | | | | | Primary B cells |
| 4D | | | | | Macrophages |
| | | | | | Dendritic cells |
| | | | | | Neutrophils |
| 7A1 | cAMP | 0.03-0.2 | μM | N/A | Activated T cells |
| 7A3 | | | | | B cells |
| | | | | | Macrophages |
| | | | | | Neutrophils |
| 8A1 | cAMP | 0.04-0.15 | μM | N/A | Activated T cells |

Additionally, more recent studies report the long term induction in activated T cells of the specific PDE isoforms, PDE1B1 (Jiang, et al., Proc Natl Acad Sci USA, vol. 93:11236-11241 (1996)), PDE1B2 (Bender, et al., Proc Natl Acad Sci USA, vol. 102:497-502 (2005)), PDE4A4 (Jiang, et al., Cell Biochem Biophys, vol. 28:135-160 (1998)), PDE4D1/D2 (Jiang, et al., Cell Biochem Biophys, vol. 28:135-160 (1998)), PDE4D3 (Jiang, et al., Cell Biochem Biophys, vol. 28:135-160 (1998)), PDE7A1 (Li et al., Science, vol. 283:848-851 (1999)), PDE7A3 (Glavas et al., Proc Natl Acad Sci USA, vol. 98:6319-6324 (2001)), and PDE8A1 (Glavas et al., Proc Natl Acad Sci USA, vol. 98:6319-6324 (2001)). Further, translocation of PDE4A4, PDE4B2, and PDE4D1/D2 to lipid rafts following T cell activation was also reported recently (Abrahamsen et al., J Immunol, vol. 173:4847-4858 (2004)). As described herein, PDE8 was identified as an additional and novel target for inhibition of chemotaxis of activated splenocytes (FIGS. 4, 5 and 7) (Dong, et al., Biochem Biophys Res Commun, vol. 345:713-719 (2006)). Thus, the fundamental concern with selective PDE4 inhibitors is that different constitutive or induced PDE isoforms in T cells control cAMP levels and reduce the efficacy of PDE4 inhibitors in vivo or require treatment doses that lead to significant side effects. A possible strategy to overcome these limitations is to inhibit other PDE isoforms that are expressed in immune cells in the hope that therapeutic activity can be retained while reducing the side effects. In order to take this approach, a complete analysis of PDE expression patterns and functions in inflammatory cells is necessary. As a first approach to address this question, the expression of splenocyte mRNA for PDE4B2, PDE7A1, and PDE8A1 following Con A stimulation of mouse splenocytes was investigated (FIG. 7). However, since these experiments were performed with bulk splenocyte cultures activated by mitogen, it remains unresolved whether the results were unique for this cell population and stimulation condition, and whether or not the same PDE isoform spectrum is expressed in purified inflammatory effector T cells. Based on these preliminary studies, this question is resolved in vitro by analyzing expression of PDE1B, PDE3B, PDE4A, B, D, PDE7A and PDE8A, B in anti-CD3 and anti-CD28 stimulated T cells.

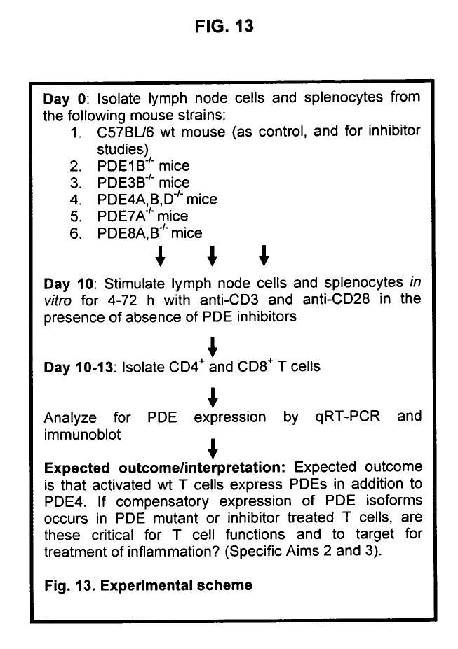

This experiment is carried out to determine in vitro expression of PDE1B, PDE3B, PDE4A,B,D, PDE7A and PDE8A, B isoforms. Expression of PDE genes is assayed and compared in activated T cells reactive to myelin antigen with unstimulated and mitogen activated T cells, using specific primers in qRT-PCR and antibodies in immunoblot analysis. Detection of specific PDE genes and proteins is performed using the methods described above in Example 1. In order to assess the kinetics of PDE expression in T cells undergoing activation, mouse CD4⁺ and CD8⁺ T cells in lymph node cells from C56BL/6 mice are purified after in vitro stimulation with anti-CD3 and anti-CD28 mAbs at various time points (FIG. 13). From these experiments, PDE isoforms that can play a regulatory role in activated inflammatory T cell populations are identified.

In addition, studies are provided to determine compensatory PDE isoform induction in anti-CD3 and anti-CD28 stimulated T cells from PDE^{-/-} mice and cells treated with selective PDE inhibitors. To assess the expression of PDE isoforms in autoreactive T cells in response to genetic or pharmacologic modulation, compensatory changes in expression of PDEs under these experimental conditions are evaluated. In order to accomplish this goal, anti-CD3 and anti-CD4 stimulated T cells from PDE^{-/-} mice or T cells treated with selective PDE inhibitors are isolated and assayed for expression levels of PDE1B, PDE3B, PDE4A,B,D, PDE7A and PDE8A,B isoforms by qRT-PCR and Western immunoblotting. Mouse mutants used in the proposed experiments are listed in Table 2. (Yang, et al., J Immunol, vol. 171:6414-6420 (2003); Jin et al., Methods Mol Biol, vol. 307:191-210 (2005); Ariga et al., J Immunol, vol. 173:7531-7538 (2004); Choi et al., J Clin Invest 116:3240-3251 (2006); Reed et al., J Neurosci, vol. 22:5188-5197 (2002); and Vasta et al., Proc Natl Acad Sci USA, vol. 103:19925-19930 (2006)). Selective inhibitors for these and further studies are compiled in Table 3. An outline of the experimental approach is given above (FIG. 13).

TABLE 2
PDE^{-/-} mice and observed phenotype

| Disrupted Gene | Phenotype |
|---|---|
| PDE1B | ↑ locomotor activity |
| PDE3B | Insulin resistance |
| PDE4A, B, D | (4A) ↓ airway disease |
| | (4B) ↓ TNF-α 90% |
| | (4D) emesis, reduced growth |
| PDE7A | None apparent |
| PDE8A, B | None apparent |

TABLE 3
Selective PDE inhibitors used for in vitro studies

| Name | Potency (IC₅₀) | Concentration in vitro |
|---|---|---|
| Vinpocetine | PDE1 (5-25 μM) | 10 μM |
| Cilostamide | PDE3 (0.005 μM) | 3 μM |
| Rolipram | PDE4 (1 μM) | 10 μM |
| BYK308438 | PDE4B2 (204 nM) | 1 μM |
| | PDE7A1 (17 nM) | |
| Proprietary (Altana) | PDE7 (17 nM) | 1 μM |
| Proprietary (Pfizer) | PDE8 (160 nM) | 1 μM |
| Dipyridamole | PDE8 (4-9 μM) | 100 μM |

### Example 8

### Determination of Regulatory Functions of PDE Isoforms

The studies described herein are carried out to determine the regulatory functions of novel PDE isoforms in anti-CD3 and anti-CD28 stimulated T cells from wildtype and PDE mutant mice and T cells treated with selective PDE inhibitors.

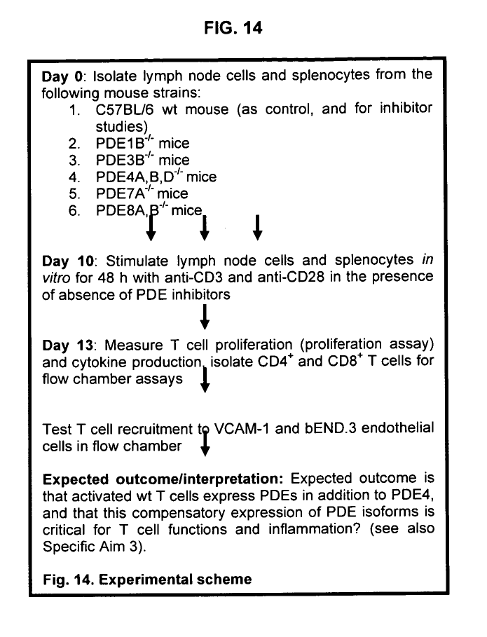

These studies test the hypothesis that if PDE isoforms are induced during T cell activation or as part of a compensatory response to genetic or pharmacologic modulation, their role can be determined in assays measuring T cell functions related to inflammation as depicted in (FIG. 14). To address this goal, test are run in specific PDE^{-/-} mice (Table 2) anti-CD3 and anti-CD28 mAb dependent T cell stimulation in proliferation and cytokine production assays. Similarly, the effect of selective PDE inhibitors is examined (Table 3). These experiments are carried out to determine which PDEs control distinct T cell effector functions, and to which extent. An additional critical T cell function addressed in these studies is T cell recruitment at a model of the microvascular endothelium. Directed migration of blood-borne cells to distinct target tissues plays an important role in numerous physiologic and pathologic conditions. Blocking leukocyte extravasation has a profound therapeutic effect on inflammatory diseases that involve recruitment of pathogenic T cells (Steinman, Cell, vol. 85:299-302 (1996); Zamvil and Steinman, Annu Rev Immunol, vol. 8:579-621 (1990); Martin, et al., Annu Rev Immunol, vol. 10:153-187 (1992); Martin et al., Nat Immunol, vol. 2:785-788 (2001); Hafler, J Clin Invest, vol. 113:788-794 (2004); Fox and Ransohoff, Trends Immunol, vol. 25:632-636 (2004); Ransohoff et al., Nat Rev Immunol, vol. 3:569-581 (2003); Feldmann and Steinman, Nature, vol. 435:612-619 (2005); Steinman, Nat Rev Drug Discov, vol. 4:510-518 (2005); and Steinman and Zamvil, Trends Immunol, vol. 26:565-571 (2005)). The efficacy of anti-migratory drugs targeting a number of different molecules has been confirmed in vitro and in vivo. Natalizumab, for example, which blocks α4β1 integrin and thereby prevents attachment of leukocytes to their endothelial ligand, vascular cell adhesion molecule-1 (VCAM-1), is now an approved drug for the treatment of MS and Crohn's disease, thus highlighting adhesion blocking antibodies as a promising avenue for the development of therapeutics effective in inflammatory diseases (Steinman, Cell, vol. 85:299-302 (1996); Zamvil and Steinman, Annu Rev Immunol, vol. 8:579-621 (1990); Martin, et al., Annu Rev Immunol, vol. 10:153-187 (1992); Martin et al., Nat Immunol, vol. 2:785-788 (2001); Hafler, J Clin Invest, vol. 113:788-794 (2004); Fox and Ransohoff, Trends Immunol, vol. 25:632-636 (2004); Ransohoff et al., Nat Rev Immunol, vol. 3:569-581 (2003); Feldmann and Steinman, Nature, vol. 435:612-619 (2005); Steinman, Nat Rev Drug Discov, vol. 4:510-518 (2005); and Steinman and Zamvil, Trends Immunol, vol. 26:565-571 (2005)). T cell homing to most target tissues requires an initial tethering step that leads to rolling in postcapillary venules followed by an activation step which triggers firm adhesion and T cell emigration (diapedesis or transendothelial migration (TEM)). This process can be divided into four distinct steps, (i.e. 1. rolling and arrest; 2. chemotactic stimulation; 3. adhesion strengthening, firm arrest; and 4. TEM). Each of these four steps involves distinct molecular pathways whose unique combination selectively enables specific subpopulations of T cells to migrate to particular organs. Efficient T cell recruitment at specific sites from blood through endothelium and ultimately, into the underlying parenchyma, requires strengthening of overall cellular adhesiveness (avidity) through precise regulation of at least one of three major integrins: α4β1, αLβ2, or α4β7. Specifically, the increase in cellular avidity under the shear stress environment of the bloodstream depends on subsecond changes in intrinsic affinity and the number (valency) of the integrin-integrin ligand bonds, as well as the cytoskeletal anchorage of the integrin molecule. At the vasculature, the required signals for integrin strengthening are provided by ligand engagement and immobilized chemokines. Regulation of T cell functions by cAMP, including adhesion, polarization and chemotaxis, is well documented. Early work demonstrated that cAMP, acting through PKA, inhibits chemoattractant-triggered integrin-dependent leukocyte adhesion, thus establishing an important role for the cAMP-PKA pathway in modulating T cell recruitment (Laudanna, et al., J Biol Chem, vol. 272:24141-24144 (1997)).

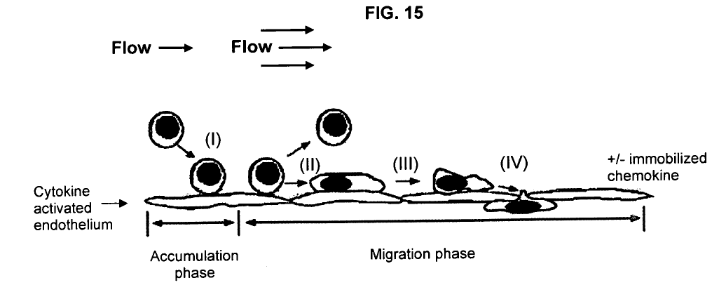

Determination of the PDE isoform control of T cell activation and function is accomplished using proliferation assay, cytokine assays, intracytoplasmic cytokine staining by flow cytometry and T cell recruitment assay in flow chamber system. Function of defined PDEs in regulating T cell activity is determined by testing anti-CD3 and anti-CD28 stimulated T cells in proliferation and cytokine production assays (Preller et al., J Immunol, vol. 178(7):4632-40 (2007)). The role of PDE1B, PDE3B, PDE4A,B,D, PDE7A and PDE8A,B genes in regulating cAMP-PKA-dependent vascular T cell recruitment is determined by real time videomicroscopy measuring rolling and arrest, activation and adhesion strengthening and transendothelial migration under physiologic shear stress in vitro (FIG. 15) (Cinamon and Alon, Methods Mol Biol, vol. 239:233-242 (2004); Cinamon and Alon, J Immunol Methods, vol. 273:53-62 (2003); Cinamon et al., Nat Immunol, vol. 2:515-522 (2001); and Shamri et al., Nat Immunol, vol. 6:497-506 (2005)). Specifically, proliferation and cytokine production of anti-CD3 and anti-CD28 stimulated T cells are performed as described for antigen-specific T cells).

In order to determine the role of PDEs in regulating T cell recruitment at brain derived endothelial cells, the studies of adhesion and chemotaxis are extended by analyzing T cell attachment to VCAM-1 and endothelial cells and TEM in a flow chamber model. During extravasation, T cells must adhere to and migrate across the endothelial barrier under the shear stress environment created by moving blood (Cinamon and Alon, Methods Mol Biol, vol. 239:233-242 (2004); Cinamon and Alon, J Immunol Methods, vol. 273:53-62 (2003); Cinamon et al., Nat Immunol, vol. 2:515-522 (2001); and Shamri et al., Nat Immunol, vol. 6:497-506 (2005)). Most migration studies have been performed under static condition in modified Boyden chambers such as Transwell assays. While T cells will migrate across a barrier in response to a chemokine gradient, it is unknown if the gradient mimics the in vivo chemokine distribution along the endothelial surface. Furthermore, chamber assays are performed under non-physiological timeframes. Intravital microscopy has predicted the window of T cell extravasation to be on the order of min, yet static migration assays are analyzed in h. Continuous shear stress in vitro promotes rapid and efficient transmigration of lymphocytes which is dependent on integrins, an intact actin cytoskeleton, and chemokine signaling, thus providing a physiologically relevant framework in which to interpret T cell recruitment. The α4β1 integrin/VCAM-1 interaction is involved in each of the steps leading to recruitment of T cells to the CNS, while globally increasing cAMP results in decreased T cell adhesion and motility. It remains to be determined how tight regulation of cAMP may differentially affect the distinct recruitment steps. Several PDEs are known to be compartmentalized intracellularly and thus degrade cAMP in a spatially restricted manner. As a result of this, even PDEs less globally abundant than PDE4 may well be functionally critical. While the role of PDE4 in T cell migration has been addressed, none of these experiments has been performed under physiologic shear stress conditions. Therefore, w the role of all PDEs expressed in T cells in addition to PDE4 is determined using the flow chamber assay system.

### Example 9

### Identifying Unique and Overlapping Functions of PDE1, PDE3, PDE4, PDE7 and PDE8 Gene Families During Inflammation

Studies are provided herein to determine the effect of non-PDE4 selective PDE inhibitors in the treatment of established inflammation. For example, knock-out of specific PDE genes and injection of selective PDE inhibitors could control T cell proliferation or cytokine production and migration, but fail to suppress inflammatory T cells in vivo. Therefore, studies are carried out to determine the susceptibility of specific PDE^{-/-} mice to inflammation in vivo. For this purpose, active EAE is induced, and this model is used to test therapeutic efficacy of selective PDE inhibitors to abolish or ameliorate established clinical signs of this inflammatory disease (Giembycz, Curr Opin Pharmacol, vol. 5:238-244 (2005); Smith and D. Spina, Curr Opin Investig Drugs, vol. 6:1136-1141 (2005); Dyke and Montana, Expert Opin Investig Drugs, vol. 11:1-13 (2002); Huang, et al., Curr Opin Chem Biol, vol. 5:432-438 (2001); Essayan, Biochem Pharmacol, vol. 57:965-973 (1999); Kroegel and Foerster, Expert Opin Investig Drugs, vol. 16:109-124 (2007); Folcik et al., J Neuroimmunol, vol. 97:119-128 (1999); and Dinter et al., J Neuroimmunol, vol. 108:136-146 (2000)). EAE is used because it represents a well-characterized model system to test anti-inflammatory therapies in vivo (Steinman, Cell, vol. 85:299-302 (1996); Zamvil and Steinman, Annu Rev Immunol, vol. 8:579-621 (1990); Martin, et al., Annu Rev Immunol, vol. 10:153-187 (1992); Martin et al., Nat Immunol, vol. 2:785-788 (2001); Hafler, J Clin Invest, vol. 113:788-794 (2004); Fox and Ransohoff, Trends Immunol, vol. 25:632-636 (2004); Ransohoff et al., Nat Rev Immunol, vol. 3:569-581 (2003); Feldmann and Steinman, Nature, vol. 435:612-619 (2005); Steinman, Nat Rev Drug Discov, vol. 4:510-518 (2005); and Steinman and Zamvil, Trends Immunol, vol. 26:565-571 (2005); and Steinman and Zamvil, Ann Neurol, vol. 60:12-21 (2006)).

Specific PDE^{-/-} mice and wt control mice are immunized with the MOG p35-55 in CFA and PTX to induce EAE (Preller et al., J Immunol, vol. 178(7):4632-40 (2007)). Animals are observed for up to 60 days for clinical signs of EAE. Selected animals at days 5-50 and all mice at day 60 are sacrificed and the brain and spinal cord examined by H&E histology to determine whether PDE genes control EAE. As a complementary approach, C57BL/6 wt mice are immunized with MOG p35-55 to induce EAE, and the selective PDE inhibitors are injected after the onset of clinical signs of EAE (FIG. 16; usually day 10-15). Dosage of inhibitors used are listed in Table 4.

TABLE 4
Selective PDE inhibitors used in in vivo studies

| Name | Dose/day | | Vehicle | Route | Specificity |
|---|---|---|---|---|---|
| Vinpocetine | 3 | mg/kg | PBS and | i.p. | PDE1 |
| | | | Tween20 | | |
| Cilostamide | 10 | mg/kg | PBS | i.p. | PDE3 |
| Rolipram | 1-6.25 | mg/kg | PEG, saline | i.p. | PDE4 |
| Proprietary | 1 | mg/kg | DMSO | i.p. | PDE7 |
| (Altana) | | | | | |
| Proprietary | 1 | mg/kg | DMSO | i.p. | PDE8 |
| (Pfizer) | | | | | |
| Dipyridamole | 100 | mg/kg | DMSO | i.p. | PDE8 |

In initial experiments, animals are treated for a period of 10, 20 and 30 days to determine whether non-PDE4 selective PDE inhibitors treat established EAE, and whether this is a long-term treatment effect or requires continuous application of PDE inhibitor. EAE is induced, and then studies are carried out to test whether PDE4 and non-PDE4 selective inhibitors treat EAE in an additive or synergistic fashion. This is tested by injection of selective PDE4 inhibitors with each one of the selective PDE1, PDE3, PDE7 and PDE8 inhibitors for the treatment of EAE.

Objective outcome measure is clinical EAE score and histopathology of CNS tissue. C57BL/6 wt mice are susceptible to active EAE. It has been established treatment of C57BL/6 mice with dipyridamole, a PDE8 inhibitor (FIG. 7). If PDE mutant mice, as expected for PDE4^{-/-} and PDE8^{-/-} mice, display altered clinical course of EAE as compared to wt control mice, their peripheral immune system is examined by quantifying CD4+ and CD8⁺ T cell populations in the spleen and lymph nodes. This alternative approach is used for any mutant strain with an EAE phenotype. The studies described above are carried out to indicate the composition of peripheral T cell populations as attempts are made to purify T cells for the study of PDE expression and functions in vitro. Based on published reports, there is little evidence for PDE gene knock out affecting overall T cell development and compartment in mice (Yang, et al., J Immunol, vol. 171:6414-6420 (2003); Jin et al., Methods Mol Biol, vol. 307:191-210 (2005); Ariga et al., J Immunol, vol. 173:7531-7538 (2004); Choi et al., J Clin Invest 116:3240-3251 (2006); Reed et al., J Neurosci, vol. 22:5188-5197 (2002); and Vasta et al., Proc Natl Acad Sci USA, vol. 103:19925-19930 (2006)). If it is determined that T cells from PDE^{-/-} mice or inhibitor treated T cells fail to display any obvious T cell dysfunctions in vitro in the studies described above, it is still possible for PDE gene knock out or PDE inhibitors to prevent or treat EAE, for example by immune deviation or induction of suppressive or regulatory T cells (Tregs) in vivo. This possibility is supported by a recent report indicating signals required to induce and maintain Tregs include Foxp3-dependent repression of PDE3B (Gavin, et al., Nature, ePublication, (Jan. 14, 2007); vol. 445(7129):771-5 (2007)). If the studies described above produce diverging results from in vitro and in vivo studies, additional studies are carried out to address mechanisms of tolerance induction (Steinman, Cell, vol. 85:299-302 (1996); Zamvil and Steinman, Annu Rev Immunol, vol. 8:579-621 (1990); Martin, et al., Annu Rev Immunol, vol. 10:153-187 (1992); Martin et al., Nat Immunol, vol. 2:785-788 (2001); Hafler, J Clin Invest, vol. 113:788-794 (2004); Fox and Ransohoff, Trends Immunol, vol. 25:632-636 (2004); Ransohoff et al., Nat Rev Immunol, vol. 3:569-581 (2003); Feldmann and Steinman, Nature, vol. 435:612-619 (2005); Steinman, Nat Rev Drug Discov, vol. 4:510-518 (2005); and Steinman and Zamvil, Trends Immunol, vol. 26:565-571 (2005)) by PDE knock out or selective inhibition through effector T cell suppression, deviation Rocken et al., Immunol Today, vol. 17:225-231 (1996); Rocken and Shevach, Immunol Rev, vol. 149:175-194 (1996); and Racke et al., J Exp Med, vol. 180:1961-1966 (1994), deletion or energy, or generation of Tregs (Qiao, et al., Immunology, vol. 120(4):447-55 (2007); and Shevach et al., Immunol Rev 212:60-73 (2006)).

### Example 10

### Effect of Targeting PDE8 on Dosage Requirements for PDE4 Inhibition

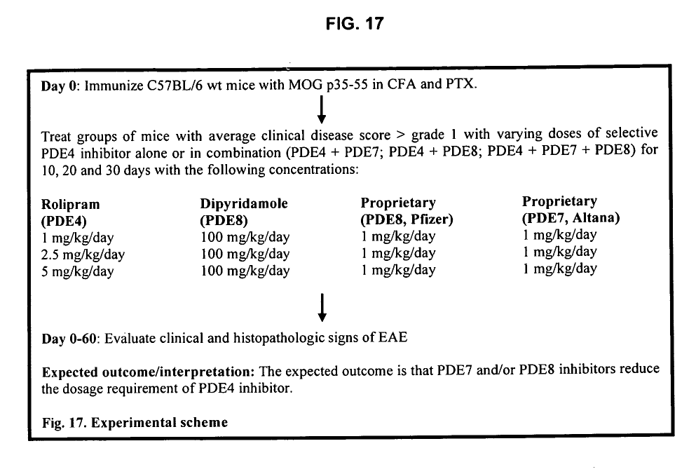

The efficacy of rolipram in reversing established EAE and preventing EAE progression is unclear (Moore et al., J Pharmacol Exp Ther 319:63-72 (2006)). Several selective PDE4 inhibitors, including cilomilast and roflumilast, are in clinical trials for the treatment of chronic obstructive pulmonary disease. Despite some encouraging data from these phase III clinical trials, the current generation of PDE4 inhibitors is hampered by a low therapeutic index. A major obstacle is their propensity to evoke side effects, of which nausea, diarrhea, abdominal pain, vomiting, increased gastric secretions, and dyspepsia are the most common (Burnouf and Pruniaux. Curr Pharm Des 8:1255-1296 (2002)). Therefore, the therapeutic potential of inhibiting PDE isoforms additional to PDE4 are investigated using the studies carried out herein. One means of improving the therapeutic index and safety of PDE4 inhibitors may lie in targeting PDE isoforms in addition to PDE4 (Giembycz, Curr Opin Pharmacol 5:238-244 (2005); Giembycz, Proc Am Thorac Soc 2:326-333; discussion 340-321 (2005); Giembycz, Br J Clin Pharmacol 62:138-152 (2006); and Giembycz and Smith, Curr Pharm Des 12:3207-3220 (2006). Inhibition of PDE4 with PDE7 and/or PDE8 enhances clinical efficacy. Using inhibitors to additional PDEs that are effective in treating EAE, a combinational treatment is developed to limit the dose of PDE4 inhibitor needed, thus maintaining efficacy while limiting side effects. In order to achieve this goal, studies are carried out to address the role of PDE isoforms in CNS inflammation in vivo, using EAE as a suitable in vivo disease model. In these studies, therapeutic doses of selective PDE4 inhibitors along with PDE7 and PDE8 inhibitors are titrated in the EAE model. In the EAE model, various dosages of a PDE inhibitor, alone or in combination with other PDE inhibitors, are administered as shown in FIG. 17.

### Example 11

### Inhibition of Metastatic Cancers Using Selective PDE Inhibitors

The studies below are carried out in relation to breast cancer metastases; however, the methods and compositions used herein are also useful in the treatment and/or inhibition of a wide variety of metastatic cancers.

Deaths from breast cancer almost always arise from metastasis of the transformed cells to other sites in the body (Steeg Nat Med, 12: 895-904, 2006). Hence, uncovering a means of inhibiting breast cancer metastasis would provide a significant advance in the treatment of this disease. Studies in cell lines and animals have shown that breast cancer cell growth and migration can be inhibited by cAMP (Marko et al., Chem Res Toxicol, 13: 944-948, 2000; O'Connor et al., J Cell Biol, 143: 1749-1760, 1998; and O'Connor et al., J Cell Biol, 148: 253-258, 2000). Selective elevation of cAMP in breast cancer cells is, therefore, an effective means to treat this disease, either alone, or in combination with other established treatments. A principal means of selectively elevating cAMP and activating the cAMP signaling pathway in a given tissue type is through inhibition of selective form(s) of cAMP phosphodiesterase (PDE) present in that tissue. With the existence of multiple transcription initiation sites, as well as alternatively spliced forms of many of these PDE genes, more than 100 different forms of PDE have been identified, many of which have been shown to vary in their expression in different tissues, intracellular localization, and interaction with different intracellular signaling pathways (Lerner et al., Biochem J, 393: 21-41, 2006; Bender and Beavo, Pharmacol Rev, 58: 488-520, 2006; Soderling and Beavo, Curr Opin Cell Biol, 12: 174-179, 2000; and Francis et al., Prog Nucleic Acid Res Mol Biol, 65: 1-52, 2001). Where PDE expression has been examined in human breast cancer cell lines, such as MCF-7, by activity analysis, results show PDE4 activity to be the predominant form expressed Marko et al., Chem Res Toxicol, 13: 944-948, 2000). Treatment of some breast cancer cell lines with PDE4 selective inhibitors inhibits both their growth and their migration and induces them to undergo apoptosis (Marko et al., Chem Res Toxicol, 13: 944-948, 2000; O'Connor et al., J Cell Biol, 143: 1749-1760, 1998; and O'Connor et al., J Cell Biol, 148: 253-258, 2000). The PDE4 gene family consists of four homologous, but distinct genes, encoding at least 20 different splice variants (Houslay, Drug Discov Today, 10: 1503-1519, 2005). Several recent studies have shown that the chemokine, CXCL12, acting through its cognate receptor, CXCR4, regulates the directional trafficking and invasion of breast cancer cells to sites of metastasis (Luker and Lucker, Cancer Lett, 238: 30-41, 2006; Smith et al., Cancer Res, 64: 8604-8612, 2004; Fernandis et al., Oncogene, 23: 157-167, 2004; and Lee et al., Mol Cancer Res, 2: 327-338, 2004). A recent study has shown that the newly discovered PDE gene, PDE8A, regulates the CXCL12-induced chemotaxis of activated lymphocytes, and that it is necessary to inhibit PDE8, as well as PDE4, in order to inhibit CXCL12-directed chemotaxis of activated lymphocytes (Dong, et al., Biochem Biophys Res Commun, 345: 713-719, 2006). Hence, PDEs provide excellent targets for inhibiting breast cancer metastasis. The studies herein analyze PDE expression in cultured human breast cancer cells, and through selective inhibition of these PDE forms, using gene family-specific PDE inhibitors and RNAi techniques, to identify targets for inhibiting migration of the breast cancer cells.

The studies herein test the hypothesis that PDE inhibitors, acting through PKA-dependent phosphorylation and inhibition of RhoA, inhibit breast cancer cell motility, and thereby inhibit breast cancer metastasis. Breast cancer cell motility is dependent upon activation of the small GTPase protein, RhoA, and stress fiber formation. RhoA is inhibited upon phosphorylation by cAMP-dependent protein kinase (PKA), following stimulation of the cAMP signaling pathway. Stimulation of the cAMP signaling pathway is achieved by inhibiting the form(s) of PDE expressed in breast cancer cells. The effects of this stimulation on breast cancer motility is analyzed.

These studies analyze the expression of different forms of PDEs in breast cancer cells, using both estrogen receptor positive and negative cell lines, and 2) examine the effect of selective inhibition or suppression of the expression of specific PDE forms expressed in these breast cancer cell lines for their effect on a) migration of the cells, b) formation of GTP-activated RhoA, and c) stress fiber formation. Analysis of cAMP PDE isoforms expressed in breast cancer cell lines is determined both by quantitative real-time RT-PCR and Western immunoblotting, using antibodies specific to each of the known cAMP PDE genes. Inhibition of the expressed forms of PDE with selective PDE gene-family inhibitors, or suppression of their expression with RNAi, is examined for their effects on migration of the cells, GTP-activated RhoA formation, and stress fiber formation.

To analyze the expression of different forms of PDEs in breast cancer, two breast cancer cell lines, MCF-7, an estrogen receptor-positive cell line, and MDA-MB-231, an estrogen receptor-negative cell line, are used. Quantitative analysis of the expression of PDEs in these cell lines is determined by quantitative real-time RT-PCR (qPCR) and Western immunoblot analysis using probes and antibodies specific for the known genes of cAMP PDEs. Of the 21 known genes that encode PDEs, 5 are specific for cGMP, and 16 are capable of hydrolyzing cAMP. Analysis is concentrated on the expression of these 16 cAMP PDE genes, notably PDEs 1A, B, C, 2A, 3A, B, 4A, B, C, D, 7A, B, 8A, B, 10A, 11A. qPCR and Western immunoblot determination of PDE expression are measured by methods recently published for determination of PDE expression in hematopoietic cells (Dong, et al., Biochem Biophys Res Commun, 345: 713-719, 2006; and Tiwari et al., Biochem. Pharmacol., 69: 473-483, 2005). Antibodies for all 21 PDE genes have now been developed and are available commercially from FabGennix Inc., Shreveport, La., and where positive expression of mRNA for cAMP PDEs is seen, an examination of corresponding protein expression is as well, using antibodies purchased from this company.

To test the effects of inhibition of the expressed PDEs on migration of the breast cancer cells in culture, PDE inhibitors are tested for their effects on breast cancer cell migration in response to CXCL12 using a transwell assay system (Dong, et al., Biochem Biophys Res Commun, 345: 713-719, 2006). Dose responses and time courses are performed with the inhibitors. Initial studies focus on inhibition of PDE4, since PDE4 has been reported to be expressed in breast cancer cells, based on activity analyses and also because PDE4 inhibitors have already been shown to be targets for inhibition of breast cancer metastasis. Four very selective and very potent PDE4 inhibitors are used rolipram, RO 20-1724, piclamilast (RP73401), and roflumilast. It has been observed, for example, that piclamilast inhibits purified PDE4 with a Ki≈0.3 nM, and does not begin to inhibit any of the other PDE gene families until concentrations of more than 5 orders of magnitude higher are reached. Although these inhibitors are very selective for PDE4 relative to the other PDE gene families, they do not distinguish between the four PDE4 subtypes, PDEs 4A-D, which is most likely due to the high degree of sequence homology in the catalytic region of these PDE4 genes. Several years ago the catalytic region of PDE4B was crystallized and its structure determined by X-ray diffraction analysis (Xu et al., Science, 288: 1822-1825, 2000). Subsequent to this, an additional crystallization and structural analysis was accomplished with PDE4 bound to the inhibitor, rolipram (Xu et al., J Mol Biol, 337: 355-365, 2004). Using this published knowledge, a scaffold-based drug discovery platform based on the 3-dimensional structure of the catalytic site of PDE4B was developed to enable screening for novel inhibitors that showed specificity for inhibition PDE4B over that of the other PDE4 subtypes. This approach produced several PDE4B selective inhibitors, including PLX513, which shows 10-20-fold greater inhibitory specificity for PDE4B over PDE4A, C and D (Card et al., Nat Biotechnol, 23: 201-207, 2005). Indeed, in a recent study of highly malignant human lymphomas that overexpress PDE4B, PLX513 was dramatically more effective than rolipram in inducing apoptosis of these cells (Smith et al., Blood, 105: 308-316, 2005). Therefore, PLX513 and any other available PDE4 subtype selective inhibitors are tested in the system described herein. In addition to these PDE4 inhibitors, selective inhibitors for all other PDE gene families that expressed in breast cancer cells are also tested for the effect on cell migration as well. The effect of PDE on cell migration is evaluated by knocking down its expression with RNAi directed against it (Lynch et al., J Biol Chem, 280: 33178-33189, 2005). For those PDE gene families for which pharmacological inhibition has a positive effect on inhibiting migration, RNAi methodology is used to determine which subtypes and which expressed splice variants of a given PDE gene family are the important targets for this inhibition, as was done recently to define which splice variants of PDE4 are functionally important in regulating β-arrestin-mediated β-adrenergic receptor desensitization (Lynch et al., J Biol Chem, 280: 33178-33189, 2005).

Migration of the breast cancer cells in response to CXCL12 and lysophosphatidic acid, as compared to control cells without a stimulus, is assayed by the transwell method (Dong, et al., Biochem Biophys Res Commun, 345: 713-719, 2006), with modifications for these adherent cells as described (Bender and Beavo, Pharmacol Rev, 58: 488-520, 2006; Soderling and Beavo, Curr Opin Cell Biol, 12: 174-179, 2000). Dose responses and time courses for inhibition of this migration by inhibitors of the different PDEs expressed in the cells, added alone and in combination, is assessed. Chemotaxis assays are done in 24-well transwell plates with a pore size of 8 μm, with the lower surface of the membrane in each transwell chamber coated for 30 min with 15 μg/ml laminin-1. Cells are harvested with trypsin and rinsed in serum-free media. Lower transwell chambers contain either medium alone (control), CXCL12 (250 ng/ml), or lysophosphatidic acid (100 nM). Cells (5×10⁴) suspended in DME/BSA media are added to the upper chamber. Following 4 h incubation at 37° C., non-migrating cells are removed from the upper chamber with a cotton swab and cells that had migrated to the lower surface of the membrane are fixed with 100% methanol and stained with 0.2% crystal violet in 2% ethanol and counted.

PKA has been reported to phosphorylate and inactivate RhoA (Howe, Biochim Biophys Acta, 1692: 159-174, 2004; Qiao et al., Am J Physiol Lung Cell Mol Physiol, 284. L972-980, 2003 and Chen et al., Exp Biol Med (Maywood), 230: 731-741, 2005), and treatment of fibroblasts with the PDE4 inhibitor, rolipram, leads to a sharp decrease in the activated state of GTP-bound RhoA in the cells (Fleming et al., J Cell Sci, 117: 2377-2388, 2004). Tests are carried out to determine if PDE inhibitors cause a similar decrease in the activated state of RhoA in breast cancer cells. To determine this, breast cancer cells are plated onto laminin in the presence and absence of PDE inhibitors for 1 hr before being lysed. Lysates from these cells are incubated with a bacterially produced fusion protein, GST-C21, to bind GTP-RhoA, bound to glutathione-coupled agarose beads. The beads are washed in lysis buffer, eluted in SDS-PAGE sample buffer and the amount of bound RhoA determined by Western blotting. These experiments are carried out to determine if inhibition of specific expressed PDEs leads to prevention of the activation of RhoA in the cells.

The major driving force of migration is the extension of a leading edge protrusion or lamellipodium, the establishment of new adhesion sites at the front, cell body contraction, and detachment of adhesions at the rear. All these steps involve the assembly, the disassembly or the reorganization of the actin cytoskeleton, and each must be coordinated both in space and time to generate productive, net forward movement (Hall, Science, 279: 509-514, 1998; and Raftopoulou and Hall, Dev Biol, 265: 23-32, 2004). Therefore, if inhibition of PDE and its subsequent inhibition of RhoA are important for the inhibition of the migration of breast cancer cells, this should be reflected in an effect on the formation of stress fibers and focal adhesion structures. As such, the effect of PDE inhibitors on stress fibers and focal adhesion structures, in cells grown in the presence and absence of laminin and stimulated with CXCL12 or vehicle alone (control), is evaluated by visualizing these with fluorescent staining, respectively, of actin and vinculin. For visualizing stress fibers and focal adhesion structures, cells are fixed with 5% paraformaldehyde, washed with PBS supplemented with 100 mM glycine, then permeabilized with PBS supplemented with 0.1% saponin and 20 mM glycine. After blocking with PBS supplemented with 0.1% saponin and 10% FCS for 1 hour at room temperature, cells are incubated with primary antibody to vinculin at a dilution of 1:100 The vinculin is then visualized with a species-specific fluorescein isothiocyanate (FITC)-coupled secondary antibody (dilution 1:100; Sigma). Actin filaments are visualized with tetramethylrhodamine β-isothiocyanate (TRITC)-phalloidin (1 mg/ml for 45 minutes) (Sigma). Cells are then visualized using the microscopes in the imaging core. These experiments are carried out to determine if PDE inhibitors have effects on stress fiber and focal adhesion structure formation to account for their inhibition of breast cancer migration.

All publications and patent documents cited herein are incorporated herein by reference as if each such publication or document was specifically and individually indicated to be incorporated herein by reference.

While the invention has been described with reference to preferred embodiments, it will be understood by those skilled in the art that various changes may be made and equivalents may be substituted for the elements thereof without departing from the scope of the invention. In addition, many modifications may be made to adapt the teaching of the invention to a particular situation, population, individual or diagnostic or treatment method without departing from the essential scope thereof. Therefore, it is intended that the invention not be limited to the particular embodiments and best mode contemplated for carrying out this invention as described herein.

What is claimed is:

1. A method of inhibiting migration of an activated lymphocyte to a site of inflammation, comprising contacting said lymphocyte with a composition that preferentially inhibits cyclic nucleotide phosphodiesterase (PDE) 8.

2. The method of claim 1, wherein said lymphocyte comprises a T helper type 1 phenotype or a T helper type 17 phenotype.

3. The method of claim 1, wherein said composition comprises dipyridamole or a derivative thereof.

4. The method of claim 1, wherein said composition further comprises an inhibitor of PDE4, an inhibitor of PDE7 or both an inhibitor of PDE4 and an inhibitor of PDE7.

5. An anti-inflammatory composition comprising a synergistic combination of a PDE8 inhibitor and a PDE4 inhibitor.

6. A method of reducing or preventing a symptom associated with a disease of the immune system comprising administering to a subject in need thereof a composition comprising an inhibitor of PDE8 in an amount effective to reduce lymphocyte chemotaxis in said subject.

7. The method of claim 6, wherein said disease is an autoimmune disease or allergic disease selected from the group comprising multiple sclerosis, type 1 diabetes, rheumatoid arthritis, asthma, chronic obstructive pulmonary diseases, inflammatory bowel disease, Alzheimer's disease and other neurodegenerative diseases with inflammatory components, atherosclerosis, vasculitis, and cancer.

8. The method of claim 6, wherein said composition further comprises an inhibitor of PDE4, an inhibitor of PDE7 or both an inhibitor of PDE4 and an inhibitor of PDE7.

9. The method of claim 6, wherein said disease is multiple sclerosis and said inhibitor of PDE8 is dipyridamole or a clinically effective derivative thereof.

10. A method of modulating an inflammatory response in a subject comprising administering to a subject in need thereof a composition comprising an inhibitor of PDE8 in an amount effective to modulate lymphocyte chemotaxis in said subject.

11. The method of claim 10, wherein said composition further comprises an inhibitor of PDE4, an inhibitor or PDE7 or both an inhibitor of PDE4 and an inhibitor of PDE7.

12. The method of claim 10, wherein said inhibitor of PDE8 is dipyridamole or a clinically effective derivative thereof.

13. A method of treating a disease associated with lymphocyte chemotaxis, lymphocyte adhesion to endothelial cells, or lymphocyte transendothelial migration comprising administering to a subject in need thereof a composition comprising an inhibitor of PDE8 in an amount effective to reduce lymphocyte chemotaxis, lymphocyte adhesion to endothelial cells, or lymphocyte transendothelial migration in said subject.

14. The method of claim 13, wherein the disease is selected from the group comprising arthritis, diabetes, inflammatory bowel diseases and metastatic cancer or a predisposition thereto.

15. The method of claim 13, wherein said composition further comprises an inhibitor of PDE4, an inhibitor of PDE7 or both an inhibitor of PDE4 and an inhibitor of PDE7.

16. The method of claim 13, wherein said inhibitor of PDE8 is dipyridamole or a clinically effective derivative thereof.

17. A method of inhibiting migration of a metastatic cancer cell, comprising contacting said metastatic cancer cell with a composition that preferentially inhibits cyclic nucleotide phosphodiesterase (PDE) 8.

18. The method of claim 17, wherein the metastatic cancer cell is derived from epithelial tissue.

19. The method of claim 17, wherein said composition further comprises an inhibitor of PDE4, an inhibitor of PDE7 or both an inhibitor of PDE4 and an inhibitor of PDE7.

20. The method of claim 17, wherein said inhibitor of PDE8 is dipyridamole or a clinically effective derivative thereof.

21. A method of treating or delaying the progression of a metastatic cancer comprising administering to a subject in need thereof a composition comprising an inhibitor of PDE8 in an amount effective to reduce migration of metastatic cancer cells.

22. The method of claim 21, wherein the metastatic cancer is derived from epithelial tissue.

23. The method of claim 21, wherein said composition further comprises an inhibitor of PDE4, an inhibitor of PDE7 or both an inhibitor of PDE4 and an inhibitor of PDE7.

23. The method of claim 21, wherein said inhibitor of PDE8 is dipyridamole or a clinically effective derivative thereof.

24. A method of reducing the severity of an inflammatory condition, comprising administering to a subject suffering from or at risk of developing said condition a synergistic combination of a PDE4 inhibitor and a PDE8 inhibitor.

25. A method of identifying a selective PDE8 inhibitor composition, comprising contacting an activated lymphocyte with a candidate PDE inhibitory compound and detecting migration of said activated lymphocyte, wherein a reduction in migration of said activated lymphocyte compared to an unstimulated lymphocyte indicates that said candidate compound selectively inhibits a PDE8-mediated inflammation.

26. A method of identifying a PDE8 inhibitor composition, comprising contacting an activated lymphocyte with a candidate PDE inhibitory compound and detecting migration of said activated lymphocyte, wherein a reduction in migration of said activated lymphocyte in the presence of said compound compared to in the absence of the compound indicates that said candidate compound inhibits a PDE8-mediated inflammation.

27. A method of identifying a selective PDE8 inhibitor composition, comprising contacting an activated lymphocyte with a candidate PDE inhibitory compound and detecting expression of activity of PDE8 in said activated lymphocyte, wherein a reduction in said PDE 8 expression or activity compared to a PDE selected from the group consisting of PDE1, 2, 3, 4, 5, 6, 7, 9, 10, or 11 indicates that said candidate compound selectively inhibits a PDE8-mediated inflammation.
